(19) United States
(12) Patent Application Publication
(10) Pub. No.: US 2007/0249808 A1
(43) Pub. Date: Oct. 25, 2007

(54) Multiple Sclerosis-related superantigen

(76) Inventors: Bernard Conrad, Geneve (CH); Bernard Mach, Chambesy (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/811,964

(22) Filed: Jun. 12, 2007

Related U.S. Application Data
(63) Continuation of application No. 10/133,036, filed on Apr. 26, 2002, now abandoned.
Application No. 10/133,036 is a continuation of application No. PCT/EP00/10659, filed on Oct. 31, 2000.

(51) Int. Cl. C07K 14/00

(52) U.S. Cl. 530/324; 530/326; 530/350

(57) ABSTRACT

The invention relates to a protein or peptide having superantigen (SAg) activity, said protein or peptide comprising the ENV protein of the human endogenous retrovirus HERV-W, the surface protein (SU) and transmembrane (TM) sub-units thereof, and fragments of HERV-W ENV and its subunits, particularly C-terminal fragments, which possess superantigen activity.

The present invention relates to a human endogenous retroviral superantigen associated with autoimmune disease, particularly multiple sclerosis. The invention also relates to derivatives of the superantigen, and to nucleic acid molecules encoding the derivatives. The invention further concerns methods for the diagnosis of autoimune disease, particularly multiple sclerosis, and methods for identifying substances which can be used in the therapy and prevention of these diseases.

For some autoimmune diseases such as Insulin Dependent Diabetes Mellitus (IDDM), Multiple Sclerosis, arthritis and others, it is known that a combination of genetic, environmental and possibly exogenous infectious factors may be important in precipitating disease. However, the precise roles of each of these factors remains. incompletely elucidated.

Recently, Conrad et al. (1994) provided evidence for superantigen involvement in IDDM aetiology and postulated that viruses may be the modifying agent responsible for the presence of superantigen on diabetic islets.

Perron et al (Perron et al, 1997) have recently identified a retrovirus, <<MRSV>>, which can be isolated from cells of multiple sclerosis patients. Whether the retrovirus contributes as a causative agent of multiple sclerosis or as a link in the pathogenic process, or whether it is merely an epiphenomenon, has not been identified. Using sequence homology with the pol gene of MRSV, Alliel et al. (1998) identified a full length endogenous provirus located on the long arm of human chromosome 7 (7q21-22). On the basis of the PBS t-RNA motif usually used for the classification of human endogenous retrovirus families (HERVs), this retrovirus has been designated <<HERV-W>> (W=tryptophan), although some authors use the designation <<HERV-7q>>. It has been postulated that HERV-W is involved in the pathogenesis of multiple sclerosis although no precise mechanism has been identified. To date, no superantigen activity of the retrovirus <<HERV-W>> has been reported.

Further endogenous retroviral sequences associated with autoimmune disease or with pregancy disorders have been reported in International Patent Application WO 99/02696.

It is an aim of the present invention to identify agents involved directly or indirectly in the pathogenesis of autoimmune disease, particularly Multiple Sclerosis (MS). On the basis of these agents reliable diagnostic procedures and therapeutic or prophylactic substances and compositions can be provided.

The present invention provides diagnostic procedures involving the detection of an expressed retrovirus having superantigen (SAg) function. It is thought that this retrovirus may be directly involved in the pathogenesis of MS by activation of autoreactive T-cells.

The present invention is based on the discovery, by the present inventors that the HERV-W (HERV-7q) retrovirus encodes superantigen (SAg) activity.

Superantigens (SAgs) (Choi et al, 1989; White et al, 1989) are microbial proteins able to mediate interactions between MHC Class II⁺- and polyclonal T-cells resulting in reciprocal activation (Acha-Orbea et al, 1991; Choi et al, 1991; Fleischer and Schrezenmeier, 1988). Their function is restricted by only two absolute requirements: the presence of MHC Class II on the surface of the presenting cells and the expression of one or more defined Variable (V)-β T cell receptor (TCR) chain(s) on T cells.

The potential role of SAgs in human diseases is ill-defined. Bacterial SAgs have been proposed to be associated with the pathogenesis of autoimmune disease (White et al, 1989). However, although pathogen disease associations have been described, none of these have as yet implicated a pathogen-encoded SAg (Howell et al, 1991; Paliard et al, 1991). A SAg-like activity resembling the one encoded by MMTV has been reported to be associated with herpesvirus infections (Dobrescu et al, 1995; Sutkowski et al, 1996). However, in neither of these two systems has it been demonstrated that the SAg activity is actually encoded by the infectious agent.

SAg activity has been reported in patients having Type I diabetes (Conrad et al 1994). The retroviral origin of the Sag activity has also recently been identified (Conrad et al., 1997).

In the context of the present invention, the inventors have identified that the ENV domain of HERV-W encodes superantigen activity. Expression of the SAg gives rise to preferential expansion of Vβ6.7 and/or Vβ17 and/or Vβ21.3 T-cell receptor positive T-cells, some of which may be autoreactive. Thus it is postulated that the expression of self-SAg leads to systemic activation of a sub-set of T-lymphocytes, among which autoreactive T-cells, will in turn give rise to autoimmune disease.

In the context of the present invention, the following terms encompass the following meanings:
a <<human autoimmune disease>> is defined as a polygenic disease characterised by the selective destruction of defined tissues mediated by the immune system. Epidemiological and genetic evidence also suggests the involvement of environmental factors.
a <<human endogenous retrovirus>> (HERV) is a retrovirus which is present in the form of proviral DNA integrated into the genome of all normal cells and is transmitted by Mendelian inheritance patterns. Such proviruses are products of rare infection and integration events of the retrovirus under consideration into germ cells of the ancestors of the host. Most endogenous retroviruses are transcriptionally silent or defective, but may be activated under certain conditions. Expression of the HERV may range from transcription of selected viral genes to production of complete viral particles, which may be infectious or non-infectious. Indeed, variants of HERV viruses may arise which are capable of an exogenous viral replication cycle, although direct experimental evidence for an exogenous life cycle is still missing. Thus, in some cases, endogenous retroviruses may also be present as exogenous retroviruses. These variants are included in the term <<HERV>> for the purposes of the invention. In the context of the invention, <<human endogenous retrovirus>> includes proviral DNA corresponding to a full retrovirus, comprising two LTR's, gag, pol and env, and further includes remnants or <<scars>> of such a full retrovirus which have arisen as a results of deletions in the retroviral DNA.

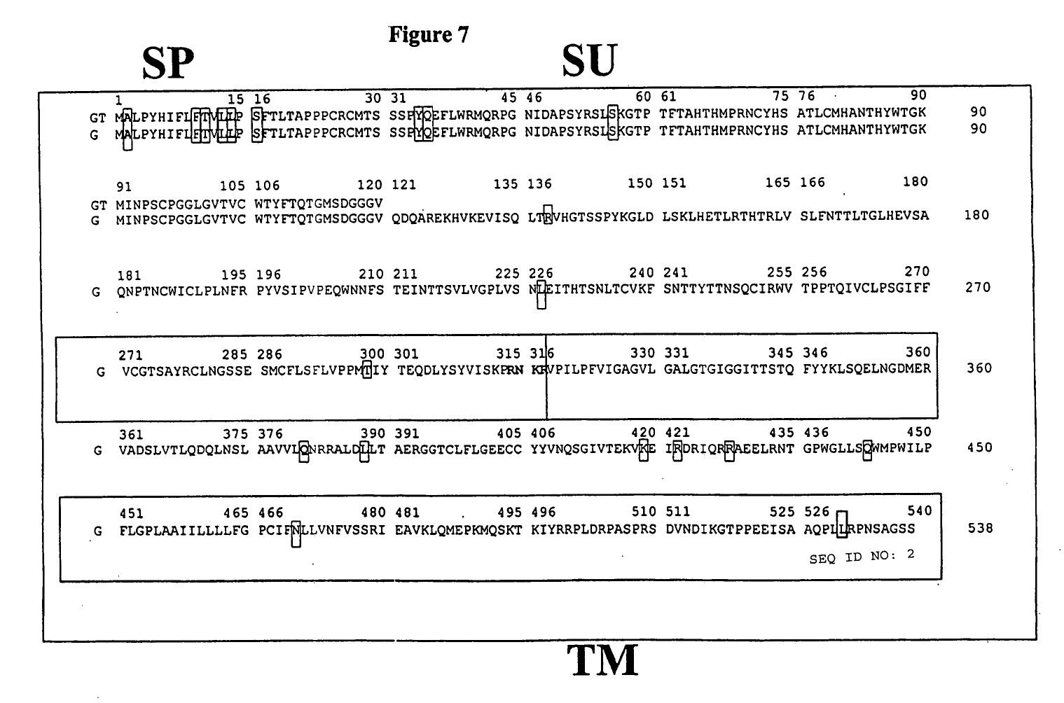

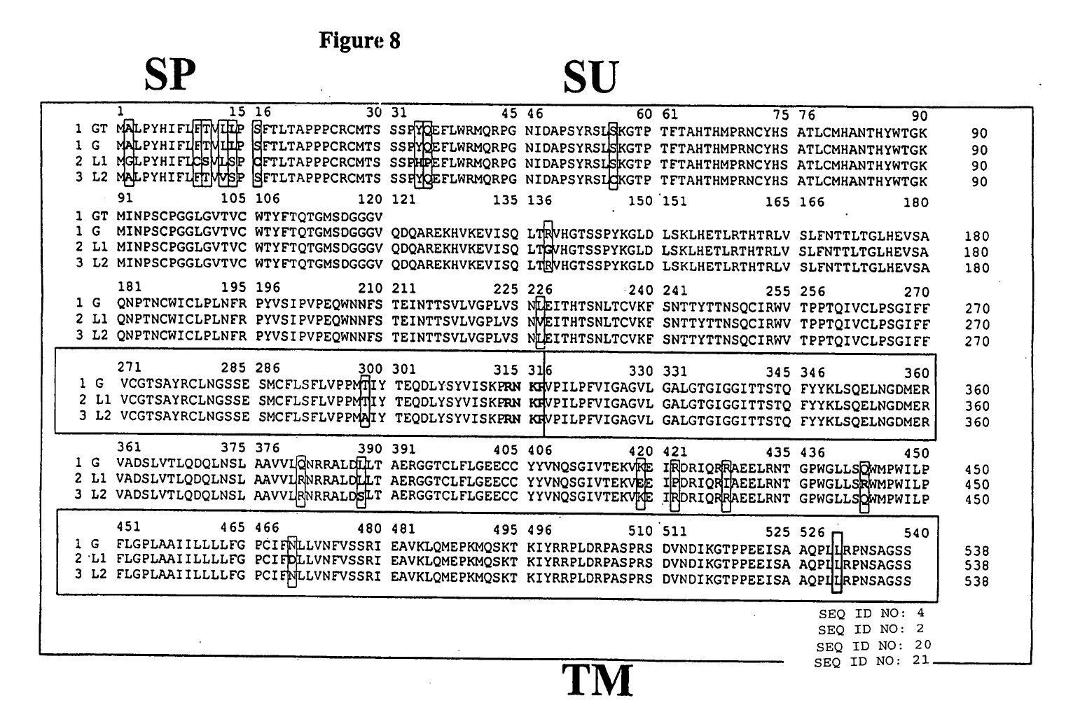

Such remnants include fragments of the typical structure, and have a minimal size of one LTR. Typically, the HERVs have at least one LTR, preferably two, and all or part of gag, pol or env.
a <<Superantigen>> or <<SAg>> is a substance, normally a protein, of microbial origin that binds to major histocompatibility complex (MHC) Class II molecules and stimulates T-cell, via interaction with the Vβ domain of the T-cell receptor (TCR). SAgs have the particular characteristic of being able to interact with a large proportion of the T-cell repertoire, i.e. all the members of a given Vβ subset or <<family>>, or even with more than one Vβ subset, rather than with single, molecular clones from distinct Vβ families as is the case with a conventional (MHC-restricted) antigen. The superantigen is said to have a mitogenic effect that is MHC Class II dependent but MHC-unrestricted. SAgs require cells that express MHC Class II for stimulation of T-cells to occur.
<<Superantigen activity>> or <<SAg activity>> signifies a capacity to stimulate T-cells in an MHC-Class II-dependent but MHC-unrestricted manner. In the context of the invention, SAg activity can be detected directly by measuring specific expansion of activated T-cells bearing a particular Vβ-chain, or indirectly in a functional assay by measuring IL-2 release by activated T-cells.
a retrovirus having SAg activity is said to be <<associated with>> an autoimmune disease, particularly MS, either when expressed retroviral RNA can be found specifically in biological samples of autoimmune patients (ie the expressed retroviral RNA is not found in individuals free of autoimmune disease), or when expressed retroviral RNA encodes a protein, having SAg activity (i.e. polymorphic or allelic forms of the retrovirus exist, only one or some of them giving rise to superantigen activity). Preferably <<associated with>> signifies in this context that retroviral SAg activation of a Vβ subset, particularly Vβ6.7 and/or Vβ17, and/or Vβ21.3 gives rise directly or indirectly to proliferation of autoreactive T-cells targeting tissue characteristic of the autoimmune disease such as MS. Blockage of SAg activity thus normally prevents generation of autoreactive T-cells. Disease <<association>> with Sag can also be defined immunologically or genetically: immunological association means that a particular disease-associated HLA haplotype is permissive for Sag, whereas resistant haplotypes are permissive for Sag inhibition. Genetic association implies a polymorphism in either the expression pattern of Sag or in the amino acid sequence of Sag, with Sag alleles exhibiting different degree of susceptibility to the disease.
cells which <<functionally express>> Sag are cells which express Sag in a manner suitable for giving rise to MHC-dependent, MHC-unrestricted T-cell stimulation in vitro or in vivo. This requires that the cell be MHC II⁺ or that it has been made MHC II⁺ by induction by agents such as IFN-γ
<<MS SAg>> or <<HERV-W SAg>> signifies Vβ6.7 and/or Vβ17 and/or Vβ21.3 specific T-cell proliferation exhibited by HERV-W ENV proteins and peptides, or derivatives. Preferably, the HERV-W ENV protein is that illustrated as protein <<G>>> in FIGS. 7 and 8, and as defined below.

In a first embodiment, the invention relates to proteins expressed by a human endogenous retrovirus having SAg activity and being associated with autoimmune disease, particularly MS.

More particularly, the invention relates to a protein or peptide having superantigen (SAg) activity, said protein or peptide comprising or consisting of the ENV protein of the human endogenous retrovirus HERV-W, the surface protein (SU) or transmembrane (TM) sub-units thereof, and fragments of HERV-W ENV and its subunits, particularly C-terminal fragments, which possess superantigen activity.

Preferably, the protein or peptide having superantigen (SAg) activity consists or comprises all or part of the Env protein of HERV-W (HERV-7q), illustrated as protein <<G>> or <<GT>> in FIGS. 7 and 8. Such proteins will be referred to herein as HERV-W SAg proteins.

Specifically, said HERV-W SAg protein or peptide comprises:
i) the amino acid sequence designated <<G>> or <<GT>>, as illustrated in FIG. 7 or FIG. 8;
ii) the surface protein portion (SU) of the polypeptide <<G>> or <<GT>> illustrated in FIG. 7 or 8, or
iii) the surface protein (SU) and transmembrane portion (TM) of the polypeptide <<G>> illustrated in FIG. 7 or 8, or
iv) the transmembrane portion (TM) of the polypeptide <<G>> illustrated in FIG. 7 or 8, or
v) a protein fragment consisting of at least 20 consecutive amino acids, and preferably at least 50, 60, 70, 80, 90 or at least 100 consecutive amino acids of protein (i), (ii), (iii) or (iv) Such fragments may contain up to approximately 500 amino acids, but generally contain between 100 and 200 or 250 amino acids.

In the context of the invention, the different portions of the Env protein are generally defined as follows, wherein the numbering of the amino acid positions corresponds to that shown in FIGS. 7 and 8:
the signal peptide (SP) extends from amino acid 1 up to amino acid 20, inclusive;
the surface protein portion (SU) extends from amino acid 21 up to amino acid 317, inclusive;
the transmembrane domain (TM) extends from amino acid 318 to amino acid 538, inclusive. The TM protein encompasses a plurality of functional domains. Amino acids 318 to approximately amino acids 340-350 correspond to the fusion peptide, which is responsible for fusion of cells expressing ENV to neighboring cells. The C-terminal twenty to thirty amino acids (approximately positions 510 to 538) anchor the TM domain into the cell membrane.

According to the invention, the HERV-W SAg protein or peptide may consist exclusively of:
i) the surface protein portion (SU) of the polypeptide <<G>> or <<GT>> illustrated in FIG. 7 or 8, or,
ii) the surface protein (SU) and transmembrane portion (TM) of the polypeptide <<G>> or <<GT>> illustrated in FIG. 7 or 8, or
iii) the transmembrane portion (TM) of the polypeptide <<G>> illustrated in FIG. 7 or 8, or
iv) a protein having at least 95%, or at least 96%, or at least 97%, or at least 98% or at least 99% homology with protein (i), (ii) or (iii), preferably at least 95% or at least 96%, or at least 97%, or at least 98% or at least 99% identity with protein (i), (ii) or (iii),
iv) a protein fragment consisting of at least 20 and preferably at least 50, or at least 80 or at least 100 consecutive amino acids of protein (i), (ii), (iii) or (iv). Such fragments may consist of up to approximately 500 amino acids, but generally consist of between 100 and 200 or 250 amino acids

Particularly preferred HERV-W SAg proteins are those having between 95% and 99% identity, for example at least 98% identity with protein (i), (ii) or (iii), for example no more than a maximum of 9 or 10 amino acid differences over the whole length of the protein of reference or the sub-unit of reference, and preferably no more than 4 or 5 amino acid differences with respect to the whole length of the protein of reference or sub-unit of reference. Most preferably, the homologous sequences show no more than 4 or 5 amino acid differences with respect to the full length sequence <<G>> of FIG. 7.

The protein or peptide of the invention may be a <<composite>> protein having SAg activity, and having the formula (I): 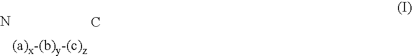
wherein
(a) is an amino acid residue, or a sequence of two or more amino acid residues,
(b) is a HERV-W SAg protein or fragment as defined above;
(c) is an amino acid residue, or a sequence of two or more amino acid residues
   <<x>>=0 or 1,
   <<z>>=0 or 1;
   <<y>>≧1, for example 1, 2, 3, 4, etc. with maximum value of 100,
   and N and C indicate amino and carboxy terminals respectively.

Such a composite protein has superantigen (SAg) activity. Component (b) is advantageously the SU or SU/TM regions of the <<G>> protein of FIG. 7 or 8, possibly in association with further amino acid sequences. The further amino acid sequences (a) and (c) do not adversely affect SAg activity, and may confer a further function on the composite protein. The resulting composite protein may be naturally occurring or artificial. When <<y>> in the above general formula has a value greater than 1, the protein may comprise a dimer, or multimer of the HERV-W SAg protein. In Formula (I), (x+z) may be greater than or equal to 1.

In Formula (I), when (b) consists of the SU or SU/TM regions of the <<G>> protein of FIG. 7 or 8, the sequence (a) if present, preferably does not consist of any one of the signal sequences:

MALPYHIFLFTVLLPSFTLT, SEQ ID NO: 5

MGLPYHIFLCSVLSPCFTLT, SEQ ID NO: 6

MALPYHIFLFTVVSPSFTLT. SEQ ID NO: 7

According to a preferred embodiment, the protein or peptide of the invention comprises a protein having the formula (II): 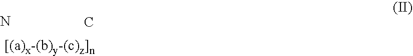
wherein
(a) is an amino acid sequence comprising or consisting of the signal sequence of the HERV-W ENV protein, or a part thereof, said part having at least five and preferably at least ten amino acids;
(b) is an amino acid sequence comprising or consisting of the SU portion of the HERV W ENV protein or a part thereof, said part having at least fifty, preferably at least one hundred and most preferably at least one hundred and fifty amino acids;
(c) is an amino acid sequence comprising or consisting of the TM portion of the HERV W ENV protein or a part thereof, said part having at least ten, preferably at least twenty and most preferably at least fifty amino acids;
   <<x>>=0 or 1,
   <<z>>=0 or 1;
   <<y>>≧1, for example 1, 2, 3, 4, etc., with a maximum value of 100, preferably 10,
   <<n>>=≧1, for example 1, 2, 3, 4, etc., with a maximum value of 100, preferably 10;
   and N and C indicate amino and carboxy terminals respectively.

Preferably, Formula (II) corresponds to a fragment of the full length SP-SU-TM HERV-W-ENV <<G>> protein as illustrated in FIGS. 7 and 8. the protein, i.e. [(a)ₓ-(b)_{y}-(c)_{z}]ₙ of Formula (II) preferably does not consist of the full length SP-SU-TM HERV-W-ENV protein as illustrated for protein <<G>> in FIGS. 7 and 8.

Most preferably, in Formula (II), the signal sequence (a), the SU portion (b) and the TM portion (c) are those illustrated for protein <<G>> or <<GT>> in FIGS. 7 and 8.

The present inventors have established that the SAg activity of the HERV-W ENV protein resides in the portion of the protein lying beyond the first N-terminal 120 amino acids, i.e. the first 120 amino acids are not essential for SAg activity. Thus, according to a preferred embodiment, the protein or peptide (b) in the above general formula (I) is a fragment consisting of a stretch of at least 50 and preferably at least 60, 70, 80, 90 or 100 consecutive amino acids comprised within amino acids 121 to 538 of the protein <<G>> illustrated in FIG. 7 or 8.

Preferred examples of the protein (b) in Formula (I) are
a fragment consisting of amino acids 121 to 538 of the protein <<G>> illustrated in FIG. 7 or 8, or
a fragment consisting of amino acids 121 to 317 of the protein <<G>> illustrated in FIG. 7 or 8, or
a fragment consisting of amino acids 121 to 350 of the protein <<G>> illustrated in FIG. 7 or 8, or
a fragment consisting of amino acids 121 to 520 of the protein <<G>> illustrated in FIG. 7 or 8.

When protein (b) is any one of the above-listed fragments consisting of a stretch of at least 50 consecutive amino acids comprised within amino acids 121 to 538 of the HERV-W ENV protein, the values of x and z in general formula (I) may be 0 or 1, for example, x may be equal to zero and consequently the fragment defined as (a) in the general formula is absent. In such a case, the N-terminus of the HERV-SAg protein is defined by amino acid 121 as illustrated in FIGS. 7 and 8. Furthermore, the integer <<y>> in formula (I) may be equal to 1 when the protein is a monomer, and is greater than 1, for example 2 to 10 or more, when the SAg protein is a multimer.

In keeping with the above, component (b) in Formula (II) can be the full SU region as illustrated for protein <<G>> in FIG. 7 or 8, or it may be amino acids 121 to 317 of this portion of the protein. In such a Formula (II) protein, the component (a) which encodes the signal peptide (SP) may be present or absent. It is preferably present in its entirety. The component (c) which encodes the TM region is, in such a Formula (II) protein, either absent, or only partially present, for example, the fragment corresponding to amino acids 318 to 350 may be present. According to this embodiment, preferred proteins therefore comprise:
amino acids 1 to 317 of the <<G>> protein illustrated in FIG. 7 or 8;
amino acids 1 to 350 of the <<G>> protein illustrated in FIG. 7 or 8;
amino acids 1 to 340 of the <<G>> protein illustrated in FIG. 7 or 8;
amino acids 1 to 520 of the <<G>> protein illustrated in FIG. 7 or 8.

The proteins of the invention may be made by synthetic or recombinant techniques. If recombinant DNA technology is used, the HERV-W SAg protein can be obtained by the following method
i) introducing a nucleic acid encoding a HERV-W ENV protein, or derivative, having SAg activity into a cell under conditions appropriate to obtain expression of the said nucleic acid,
ii) recovering the protein produced as a result of expression of the said nucleic acid.

The cells for the production of recombinant HERV-W SAg are preferably, but not necessarily, mammalian cells and may be MHC Class II⁺ or MHC Class II⁻. SAg activity can only be exhibited in cells which are MHC Class II⁺ (or which have been induced to become MHC Class II⁺), but expression of the SAg protein can be obtained in both MHC Class II⁺ and MHC Class II⁻ cells. Typical MHC Class II⁺ cells are APCs such as B-lymphocytes, monocytes, macrophages or dendritic cells. Typical MHC Class II⁻ include HeLA cells etc.

In accordance with this embodiment, a nucleic acid encoding the full length HERV-W protein depicted in FIG. 7 (including SP, SU and TM portions) or a fragment thereof, is expressed in a mammalian cell under conditions which allow correct processing, folding and possibly dimer- or multimerisation of the expression product. The proteins having SAg activity may naturally result from a premature translational stop and possibly also from a translational frameshift.

The SAg activity of the proteins or peptides according to the invention is specific for Vβ6.7- and/or Vβ17- and/or Vβ21.3-TCR chains. The inventors have established that the specificity of the HERV-W SAg activity with regard to Vβ expansion varies, within the specified spectrum, from individual to individual, reflecting the possible existence of polymorphic genetic factors and/or immunological tolerance to the SAg. At least one of Vβ6.7- and/or Vβ17- and/or Vβ21.3-TCR chains is stimulated. The most common pattern observed is the specific expansion of Vβ6.7⁺ and Vβ17⁺-T cells, although individuals showing other combinations such as Vβ21.3 and Vβ17 expansion have been identified.

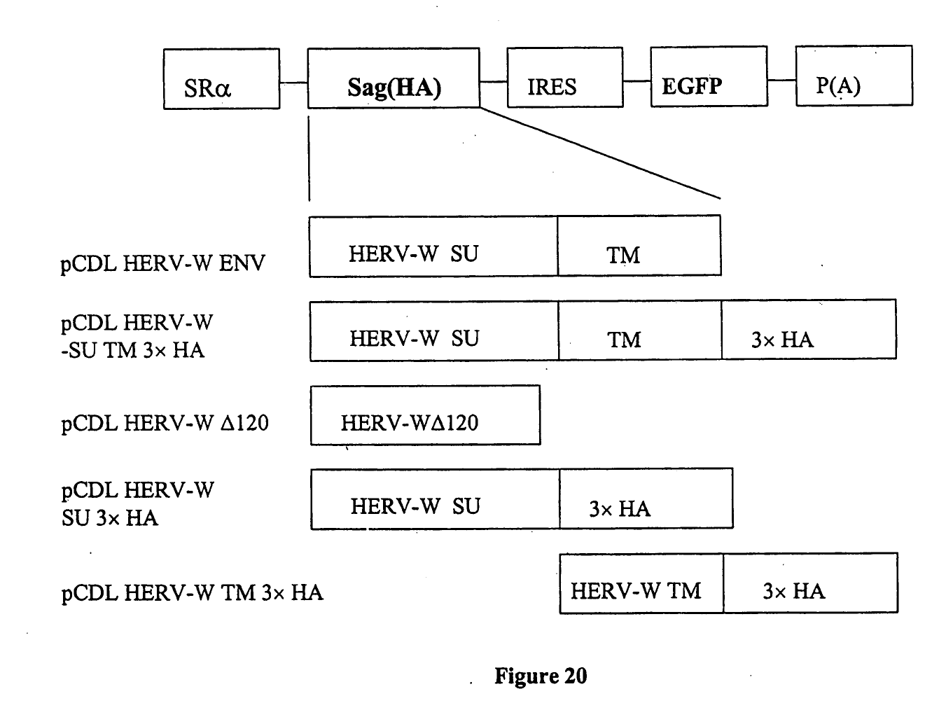

In the context of the invention, the inventors have devised a highly sensitive bicistronic assay system which is particularly adapted for measuring expression levels of transfectants expressing HERV-W SAg proteins. The bicistronic constructs are illustrated in FIG. 20. Such assays enable the detailed analysis of structure/function relationships, and allow the direct comparison of expression levels of individual constructs. Specific details of the assay are provided n the Examples below.

The invention also relates to nucleic acid molecules encoding a HERV-W SAg protein as defined above.

The nucleic acid molecule encoding HERV-W SAg activity typically corresponds to the ENV open reading frame of the retrovirus. Preferably, the nucleic acid of the invention comprises or consists of all or part of the env gene (encoding the envelope glycoprotein) of an HERV associated with MS, such as HERV-W, illustrated in FIGS. 7 and 8.

The nucleic acid of the invention may be RNA, DNA or cDNA, for example proviral DNA, or retroviral genomic RNA. Proviral DNA is naturally found integrated into the human genome. Alternatively the nucleic acid may be synthetic.

More particularly, the nucleic acid molecules of the invention have the formula (III):
5′(A)ₓ-(B)_{y}-(C)_{z}3′ (III)
wherein
(A) is a nucleotide, or an oligonucleotide of at least two nucleotides,
(B) is a nucleic acid encoding an HERV-W SAg protein;
(C) is a nucleotide, or a nucleic acid sequence of at least two nucleotides;

<<x>>=0 or 1,

<<z>>=0 or 1,

<<y>>≧1.

Preferably, in the above formula (III) the oligonucleotide (A) does not encode a peptide comprising or consisting of any one of the signal sequences:

MALPYHIFLFTVLLPSFTLT, SEQ ID NO: 5

MGLPYHIFLCSVLSPCFTLT, SEQ ID NO: 6

MALPYHIFLFTVVSPSFTLT. SEQ ID NO: 7

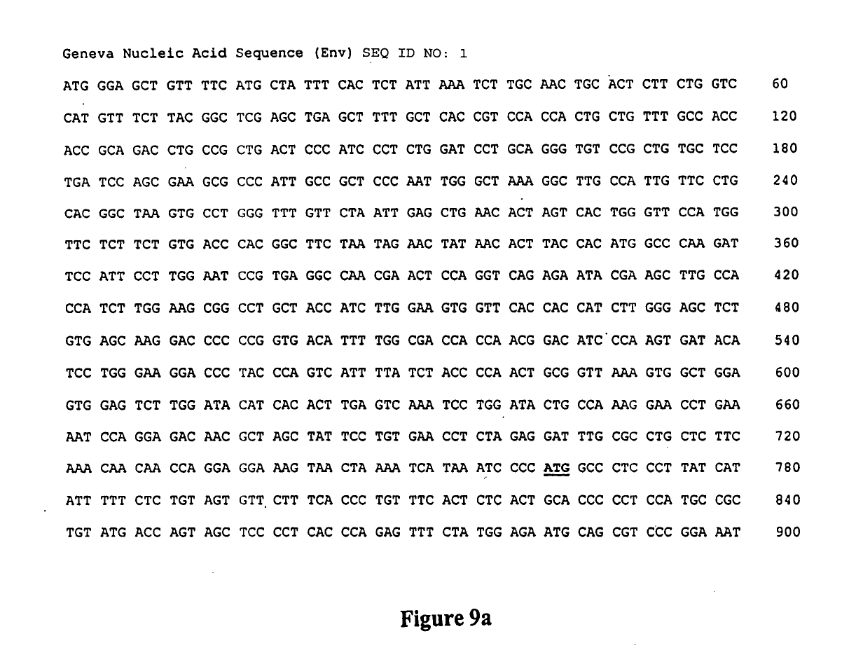

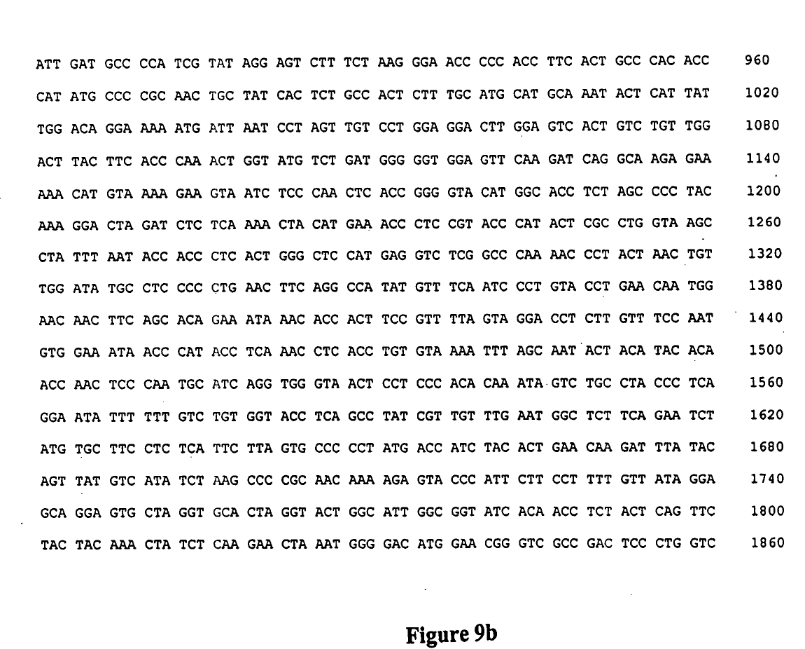

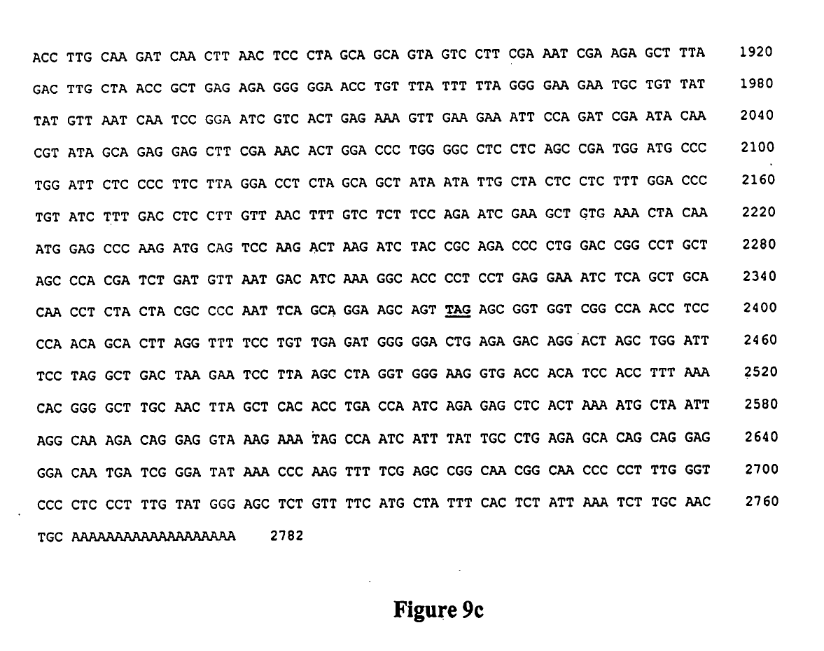

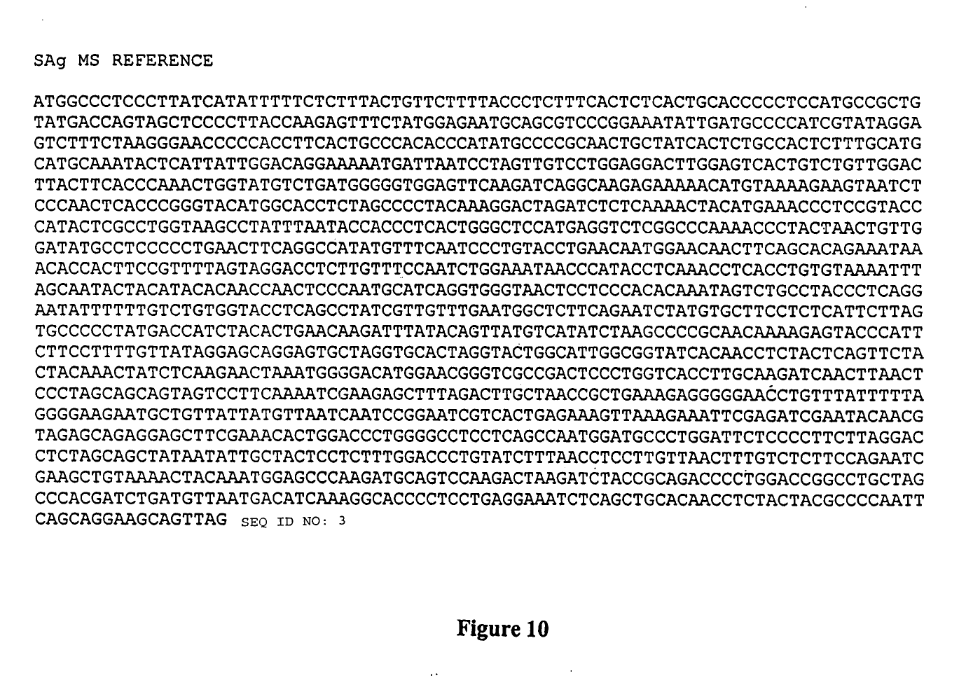

Preferred nucleic acid molecules according to the invention comprise or consist of the sequence illustrated in FIG. 9 or 10, or a fragment of either one of said sequences having at least 50 nucleotides, and preferably at least 100, and most preferably at least 300 nucleotides. Other preferred sequences are those having at least 80%, and preferably at least 90% identity with the sequence illustrated in FIG. 9 or 10, whilst still encoding SAg activity.

The nucleic acid molecules of the invention may comprise a chimeric gene wherein (A) and (C) as defined above include heterologous transcription regulatory regions operably linked to (B). By <<heterologous transcription regulatory sequences>> is meant regulatory sequences which are not those naturally used for transcription of the HERV ENV protein in the human genome.

Particularly preferred nucleic acid sequences are those encoding the proteins of Formulae (I) and (II) above, for example, encoding the following:
amino acids 121 to 538 of the protein <<G>> illustrated in FIG. 7 or 8, or
amino acids 121 to 317 of the protein <<G>> illustrated in FIG. 7 or 8, or
amino acids 121 to 350 of the protein <<G>> illustrated in FIG. 7 or 8, or
amino acids 121 to 520 of the protein <<G>> illustrated in FIG. 7 or 8.
amino acids 1 to 317 of the <<G>> protein illustrated in FIG. 7 or 8;
amino acids 1 to 350 of the <<G>> protein illustrated in FIG. 7 or 8;
amino acids 1 to 340 of the <<G>> protein illustrated in FIG. 7 or 8;
amino acids 1 to 520 of the <<G>> protein illustrated in FIG. 7 or 8.

The nucleic acid molecules of the invention further comprise sequences which are complementary to a nucleic acid molecule as defined above, for example probes, primers, ribozymes or antisense molecules to the HERV-W ENV.

Nucleic acid molecules capable of hybridizing in stringent conditions with any of the above-defined nucleic acid molecules are also within the invention. Typical stringent conditions are those where the combination of temperature and salt concentration chosen to be approximately 12-20° C. below the Tm (melting temperature) of the hybrid under study. Such nucleic acid molecules may be labelled with conventional labelling means to act as probes or, alternatively, may be used as primers in nucleic acid amplification reactions.

The invention further relates to vector comprising any of the afore mentioned nucleic acid molecules.

The present invention involves, in a further embodiment, methods of diagnosis of autoimmune disease, particularly MS, based on the one hand on the specific presence in individuals susceptible to MS, of HERV-W SAg, and nucleic acids encoding the HERV-W SAg and on the other hand on the specific expression, in MS patients, of retroviruses having SAg activity.

The methods of diagnosis of the present invention are advantageous in so far as they are highly specific, distinguishing between different polymorphic forms of the MS-associated HERV, and further distinguishing between expressed and non-expressed viral nucleic acid. These methods can thus be reliably used even if the pathological agent is a ubiquitous endogenous retrovirus. They can be carried out on easily accessible biological samples (fluids or tissue), such as blood or plasma, without extensive pre-treatment. The diagnostic methods of the invention detect either disease-specific polymorphic forms of the retrovirus, and/or disease-specific expression of the retroviral superantigen. They can thus be applied before appearance of clinical symptoms, for example on genetically predisposed individuals. This allows suitable therapy to be initiated before autoimmune destruction occurs.

More particularly, in a first embodiment, the present invention relates to a process for the diagnosis of Multiple Sclerosis (MS) by detection of disease-specific retroviral polymorphic forms, comprising:
i) contacting a sample of genomic DNA from an individual, with nucleic acid primers suitable for the amplification, in a nucleic acid amplification reaction, of all or part of the genomic locus containing the gene encoding the HERV-W SAg of the invention,
ii) performing amplification of the said genomic locus,
iii) sequencing the thus amplified nucleic acid, the presence of nucleic acid encoding a HERV-W SAg being indicative of the presence or susceptibility to, MS or other autoimmune disease.

According to this embodiment, particularly preferred amplification primers are selected from sequences flanking the HERV-W retrovirus on chromosome 7 (7q21-22). In a preferred variant, the 3′ primer corresponds to approximately 100 bases or more, of the 3′ genomic sequence immediately flanking the HERV-W 3′ LTR on chromosome 7 (see Alliel et al, 1998), and the 5′ primer corresponds to a region of approximately 100 bases or more immediately upstream of the ATG translation initiation codon of HERV-W env. For example, the 5′ primer may be selected from any 100 base stretch, or longer, within the 5′ UTR of env (approximately nucleotides 1 to 760) as illustrated in FIG. 9.

In a further embodiment, the present invention relates to a process for the diagnosis of Multiple Sclerosis (MS) by detection of disease-specific expression of SAg, comprising:
i) contacting a sample of mRNA from an individual, with nucleic acid primers suitable for the amplification, in an RNA amplification reaction, of all or part of the RNA encoding an HERV-W SAg as defined above,
ii) performing amplification of the said RNA,
iii) sequencing the thus amplified nucleic acid, the presence of nucleic acid encoding an HERV-W SAg being indicative of the presence of, or susceptibility to, MS

Specific detection of retroviral expressed mRNA is preferably carried out using nucleic acid amplification with viral specific primers which discriminate between proviral DNA and expressed RNA template. This is of particular importance since the MS associated retrovirus is an endogenous retrovirus. Indeed, it is thought that the proviral DNA is present in all human cells, whether or not the autoimmune disease is present. False positives could therefore be obtained if a detection method were used which does not distinguish between proviral DNA and transcribed mRNA.

The biological sample to be used for specific mRNA detection according to the invention may be any body fluid or tissue but is preferably plasma or blood. Normally, total RNA is extracted from the sample using conventional techniques. DNAse treatment may be carried out to reduce contaminating cellular DNA.

By performing the amplification on total RNA samples, the effects of contaminating DNA are reduced but not eliminated, even after treatment by DNAse. The method of the present invention allows selective amplification of expressed viral RNA transcripts using at least one m-RNA specific primer, for example a poly-A specific primer, even in the presence of contaminating viral DNA in the sample. The poly-A specific primer is specific for the poly-A signals present in the R-poly(A) sequences and the 3′ extremity of the retrovirus (see for example Alliel et al).

A poly-A-specific primer having from four to 25 T's for example 5 or 20 T's is particularly suitable for the purposes of the present invention.

The mRNA specific amplification requires a reverse transcriptase (RT) step, for which the poly A-specific primer is also be used.

The second primer in the mRNA-specific PCR step may be complementary to the U3 region, or other region of the retroviral genome, for example the 5′UTR of env. When the amplification product has a size of about 300 to 500 nucleotides, the conditions applied for the amplification (PCR) step are normally the following

| i) | reverse transcriptase: | 50° C. | 30 | minutes |
|---|---|---|---|---|
| ii) | amplification: | 94° C. | 2 | minutes |
| | (for a total | 94° C. | 30 | secondes |
| | of 10 cycles) | 68° C. | 30 | secondes |
| | | −1.3° C. | each cycle | |
| | | 68° C. | 45 | secondes |
| iii) | amplification: | 94° C. | 30 | secondes |
| | (for a total | 55° C. | 30 | secondes |
| | 25 cycles) | 68° C. | 45 | secondes |

The amplified material is subjected to gel electrophoresis and hybridised with suitable probes, for example generated from the U3 region.

By performing the mRNA specific detection of the invention, the presence of expressed MS retrovirus can be reliably determined in a biological sample. This can be detected well before the apparition of any clinical symptoms. The diagnosis of the invention can thus be used to detect onset of the disease process, enabling treatment to be administered before irreversible autoimmune attack occurs.

According to a particularly preferred embodiment, MS is diagnosed by a combination of the detection of the disease-specific polymorphic form, and the detection of the disease-specific SAg expression.

The invention also encompasses pro-viral specific detection of retroviral DNA, and simultaneous detection of both expressed retroviral m-RNA and proviral DNA. Specific proviral DNA detection can be used on healthy biological samples to confirm the endogenous nature of the retrovirus. the assay detecting both retroviral mRNA and proviral DNA can be used as an internal standard.

Multiple Sclerosis may also be diagnosed according to the invention by specifically detecting SAg protein expressed by the retrovirus. Preferably, the expressed protein is detected in the biological sample, such as blood or plasma, using antibodies, particularly monoclonal antibodies, specific for the said protein. A Western-like procedure is particularly preferred, but other antibody-based recognition assays may be used.

According to another embodiment of the invention, the autoimmune disease is diagnosed by detecting in a biological sample, antibodies specific for the SAg protein expressed by the MS-associated retrovirus.

Detection of antibodies specific for these proteins is normally carried out by use of the corresponding retroviral protein or fragments thereof having at least 6 amino-acids, preferably at least 10, for example 6-25 amino acids. The proteins are usually Env or fragments thereof and usually have superantigen activity. The retroviral proteins used in the detection of the specific antibodies may be recombinant proteins obtained by introducing viral DNA encoding the appropriate part of the retrovirus into eukaryotic cell and the conditions allowing the DNA to be expressed and recovering the said protein.

In the context of the present invention, the terms “antibodies specific for retroviral proteins” signifies that the antibodies show no significant cross reaction with any other proteins likely to occur in the biological sample. Generally, such antibodies specifically bind to an epitope which occurs exclusively on the retroviral protein in question. The antibodies may recognize the retroviral protein having HERV-W SAg activity as presented by the M.H.C class II molecule.

Detection of specific antibodies may be carried out using conventional techniques such as sandwich assays, etc. Western blotting or other antibody-based recognition system may be used.

According a further embodiment of the invention, the autoimmune disease is diagnosed by detecting, in a biological sample, HERV-W SAg activity specifically associated with the autoimmune disease, for example Vβ6.7 and/or Vβ 17 and/or Vβ21.3 specific proliferation. This is done by carrying out a functional assay in which a biological fluid sample containing MHC class II+ cells, for example Antigen Presenting Cells (APC) such as dendritic cells is contacted with cells bearing one or more variable β-T-receptor chains and detecting preferential proliferation of the Vβ6.7 and/or Vβ 17 and/or Vβ21.3 subset characteristic of HERV-W associated disease. This method of diagnosis may be combined with one or more of the other methods described above to maximize specificity.

The biological sample according to this variant of the invention is typically blood and necessarily contains MHC class II+ cells such as B-lymphocytes, monocytes,macrophages or dendritic cells which have the capacity to bind the superantigen and enable it to elicit its superantigen activity. MHC class II content of the biological sample may be boosted by addition of agents such as IFN-gamma.

The biological fluid sample is contacted with cells bearing the Vβ-T receptors belonging to a variety of different families or subsets in order to detect specific Vβ6.7 subset stimulation by the putative SAg, for example V-β2, 3, 5, 6.7, 7, 8, 9, 11, 12, 13, 17, 21, 22, 23. Within any one V-β family it is advantageous to use V-β chains having junctional diversity in order to confirm superantigen activity rather than nominal antigen activity.

The cells bearing the V-β receptor chains may be either an unselected population of T-cells or T-cell hybridoma. If unselected T-cells are used, the diagnostic process is normally carried out in the following manner: the biological sample containing MHC Class II+ cells is contacted with the T-cells for approximately 3 days. A growth factor such as Interleukin 2 (IL-2) which selectively amplifies activated T-cells is then added. Enrichment of a particular V-β family or families is measured using monoclonal antibodies against the TCR-β-chain. Only amplified cells are thus detected. The monoclonal antibodies are generally conjugated with a detectable marker such as a fluorochrome. The assay can be made T-cell specific by use of a second antibody, anti CD3, specifically recognizing the CD3-receptor.

T-cell hybridoma bearing defined T-cell receptor may also be used in the functional or cell-based assay for SAg activity. An example of commercially available cells of this type are given in B. Fleischer et al. Infect. Immun. 64, 987-994, 1996. Such cell-lines are available from Immunotech, Marseille, France. According to this variant, activation of a particular family of V-β hybridoma leads to release of IL-2. IL2 release is therefore measured as read-out using conventional techniques.

The invention also relates to antibodies capable of specifically recognizing a protein according to the invention. These antibodies are preferably monoclonal. Preferred antibodies are those which specifically recognize a retroviral protein having HERV-W SAg activity and which have the capacity to block HERV-W SAg activity, i.e. block Vβ6.7 and/or Vβ 17 and/or Vβ21.3 specific proliferation. The capacity of the antibody to block this SAg activity may be tested by introducing the antibody under test into an assay system comprising:
i) MHC Class II⁺ cells expressing retroviral protein having HERV-W SAg activity and
ii) cells bearing Vβ6.7-T cell receptor chains, or cells bearing Vβ 17 T cell receptor chains or cells bearing Vβ21.3 T cell receptor chains,
   and determining the capacity of the antibody under test to diminish or block Vβ-specific stimulation by the HERV-W Sag.

The steps described below involve the use of Sag-expressing transfectant cells such as those described in the examples, to inhibit the effect of Sag in vitro and in vivo.

Mabs directed against the HERV-W SAg protein (or portion of it) are generated by standard procedures used to generate antibodies against cell surface antigens. Mice are immunized with mouse cells expressing both Sag and MHC class II (such as a Sag-transfected mouse B cell line described in the examples below). After fusion with hybridoma cell lines, supernatants are screened for the presence of anti-Sag antibodies on microtiter plates for reactivity to Sag transfectants cells, with non-transfected cells as negative controls. Only Mabs with reactivity specific for Sag expressing cells are selected.

All such Mabs, either as culture supernatants or as ascites fluid, are then tested for their ability to block the Sag activity, as assayed by the T cell assay in the presence of Sag-expressing human MHC class II positive transfectants. A preferred version of this assay makes use of Vβ-specific hybridomas as T cell targets for read out. Controls are blocking of the same assay by anti-HLA-DR Mabs, which is known to inhibit the Sag effect on T cell activation. Mabs capable of efficiently blocking the Vβ-specific Sag effect, when tested at several dilutions, are selected as anti-Sag blocking Mabs.

Sufficient numbers of anti-Sag Mabs are screened in the functional assay to identify anti-Sag Mabs with optimal Sag blocking activity, in terms of T cell activation. Selected Sag blocking Mabs are then converted into their <<humanized>> counterpart by standard CDR grafting methodology. A humanized anti-Sag blocking Mab, directed against the HERV-W Sag, can then be tested clinically in patients.

The invention also relates to cells transfected with and expressing human endogenous retrovirus protein or peptide having HERV-SAG SAg activity. The cells may be preferably human cells other than the naturally occurring cells from auto-immune patients and may also include other type of eukaryotic cells such as monkey, mouse or other higher eukaryotes. The cells may be established cell-lines and are preferably MHC class II⁺, or MHC II⁺-inducible, such as β-lymphocytes and monocytes. Non-human higher eukaryotic cell-lines (e.g. mouse) stably transfected with the HERV-W Sags of the invention (as exemplified in the Examples below) have been found to specifically stimulate in vitro human Vβ6.7-T cells.

According to a particularly preferred embodiment, the cells of the invention are cells transfected with a chimeric gene encoding the HERV-W SAg as described above. Again, these cells are usually MHC Class II+ or MHC Class II-inducible, and have the capacity to exhibit SAg activity, specific for Vβ6.7 and/or Vβ17 and/or V21.3-TCR chains.

The invention also relates to a transgenic animal model for HERV-W-associated disease such as MS. The transgenic animal is made according to conventional techniques and includes in its genome, nucleic acid encoding the HERV-W Sags of the invention.

A further important aspect of the invention relates to the identification of substances capable of blocking or inhibiting HERV-W SAg activity. These substances are used in prophylactic and therapeutic treatment of HERV-W associated disorders such as MS.

The invention thus concerns methods for treating or preventing HERV-W associated disorders such as MS, by administering effective amounts of substances capable of blocking HERV-W Sag activity. The substances may be antibodies, proteins, peptides, derivatives of the HERV, derivatives of the Sag or small chemical molecules. The invention also relates to pharmaceutical compositions comprising these substances in association with physiological acceptable carriers, and to methods for the preparation of medicaments for use in therapy or prevention of autoimmune disease using these substances.

Further, this aspect of the invention includes a process for identifying substances capable of blocking or inhibiting HERV-W SAg activity of, comprising introducing the substance under test into an assay system comprising:
i) MHC Class II⁺ cells functionally expressing retroviral protein having HERV-W activity and;
ii) cells bearing Vβ6.7-T cell receptor chains, or Vβ17-T cell receptor chains or Vβ21.3 T cell receptor chains, and determining the capacity of the substance under test to diminish or block Vβ-specific stimulation by the HERV SAg,

The cells bearing the β-T cell receptors and the MHC Class II+ cells may be those described earlier. Read-out is IL-2 release.

The substances tested for inhibition or blockage of Sag activity in such screening procedures may be proteins, peptides, antibodies, small molecules, synthetic or naturally occurring, derivatives of the retroviruses themselves, etc. . . . Small molecules may be tested in large amounts using combinatorial chemistry libraries.

The screening procedure may include an additional preliminary step for selecting substances capable of binding to retroviral protein having HERV-W SAg activity. This additional screening step comprises contacting the substances under test, optionally labelled with detectable marker with the retroviral protein having SAg activity and detecting binding.

The HERV-W Sags of the invention or a portion thereof may be used for the identification of low molecular weight inhibitor molecules as drug candidates.

The rationale is that because HERV encoded Sags are the product of ancient infectious agents, they are not indispensable to humans and can thus be inhibited without adverse side effects.

Inhibitors of HERV-W Sag, as potential drug candidates, are preferably identified by a two step process:

In the first step, compatible with large scale, high throughput, screening of collections (<<libraries>>) of small molecular weight molecules, the recombinant HERV-W Sag protein (as defined in Claims 1 to 5) is used in a screening assay for molecules capable of simply binding to the HERV-W Sag protein (=<<ligands>>). Such high throughput screening assays are routinely performed by companies such as Novalon Inc or Scriptgen Inc, and are based either on competition for binding of peptides to the target protein or on changes in protein conformation induced by binding of a ligand to the target protein. Such primary high throughput screening for high affinity ligands capable of binding to a target recombinant protein are available commercially. This screening method requires that the HERV-W SAg protein, be available.

In the second step, any low molecular weight molecule identified as described above as capable of binding to the Sag protein, is tested in the functional Sag assay consisting of human MHC class II positive Sag transfectants and responding Vβ-specific T cells (preferably hybridomas), as described herein. Positive control for Sag inhibition is an anti-HLA-DR Mab, known to inhibit the Sag effect. All candidate molecules are thus tested, at different concentrations, for a quantitative assessment their anti-Sag inhibitory efficacy.

Compounds exhibiting anti HERV-W-Sag inhibitory effects are then tested for obvious toxicity and pharmacokinetics assays, in order to determine if they represent valuable drug candidates.

Once a substance or a composition of substances has been identified which is capable of blocking or inhibiting SAg activity, its mode of action may be identified particularly its capacity to block transcription or translation of SAg encoding sequences. This capacity can be tested by carrying out a process comprising the following steps:
i) contacting the substance under test with cells expressing retroviral protein having HERV-W SAg activity, as previously defined, and
ii) detecting loss of HERV-W SAg protein expression using SAg protein markers such as specific, labelled anti-SAg antibodies.

The antibodies used in such a detection process are of the type described earlier.

The invention also relates to a kit for screening substances capable of blocking HERV-W SAg activity of an endogenous retrovirus associated with an autoimmune disease, or of blocking transcription or translation of the retroviral SAg protein. The kit comprises:
MHC Class II⁺ cells transformed with and expressing retroviral SAg according to the invention;
cells bearing Vβ6.7 or Vβ17 or Vβ21.3-T cell receptor chains;
means to detect specific Vβ stimulation by HERV-W SAg;
optionally, labelled antibodies specifically binding to the retroviral SAg.

According to a further important aspect of the invention, there is provided a protein or peptide derived from an autoimmune related retroviral SAg as previously defined wherein the protein is modified so as to be essentially devoid of SAg activity, thereby no longer being capable of significantly activating auto-reactive T-cells. Such modified proteins are however capable of generating an immune response against SAg, the immune response involving either antibodies and/or T-cells responses. The immunogenic properties of the modified proteins are thus conserved with respect with the authentic SAg.

Such modified immunogenic proteins may be obtained by a number of conventional treatments of the SAg protein, for example by denaturation, by truncation or by mutation involving deletion, insertion or replacement of amino acids. Modified SAg proteins being essentially devoid of SAg activity but capable of generating an immune response against SAg include the truncations of the SAg protein, either at the amino or carboxyterminal, and may involve truncations of about 5-30 amino acids at either terminal.

These proteins are used in the framework of the invention as vaccines, both prophylactic and therapeutic, against HERV-W associated disorders such as MS. The vaccines of the invention comprise an immunogenically effective amount of the immunogenic protein in association with a pharmaceutically acceptable carried and optionally an adjuvant. The use of these vaccine compositions is particularly advantageous in association with the early diagnosis of MS using the method of the invention. The invention also includes the use of the immunogenic proteins in the preparation of a medicament for prophylactic or therapeutic vaccination against MS.

The rationale behind this prospective immunization technique is that because HERV encoded Sags are the product of ancient infectious agents, they are not indispensable to humans and can thus be inhibited without adverse side effects.

Identification of suitable anti-sag vaccine proteins or peptides can be made in the following way. Modified forms of the original active HERV-W Sag protein, including truncated or mutated forms, or even specific peptides derived from the Sag protein, are first tested in the functional Sag assays described above to confirm that they have lost all Sag activity (in terms of T cell activation). These modified forms of Sag are then used to immunize mice (or humans) by standard procedures and with appropriate adjuvants. Extent and efficacy of immunization is measured, including circulating anti-Sag antibodies. In a preferred example, eliciting a B cell immune response, by selecting B cell epitopes from the Sag protein as immunogen, is deliberately aimed at.

Successfully immunized animals are then tested for the effect of Sag in vivo by a standard assay, namely the injection of MHC class II positive Sag transfectants (such as the transfectants described in the examples below), known to induce in vivo a Vβ-specific T cell activation. Successful immunization against a Sag protein is expected to result in a reduction or in a block of the in vivo Sag-induced T cell activation and proliferation in effectively immunized individuals. This procedure is referred to as anti-Sag vaccination.

The vaccines of the invention can be prepared as injectables, e.g. liquid solutions or suspensions. Solid forms for solution in, or suspension in, a liquid prior to injection also can be prepared. Optionally, the preparation also can be emulsified. The active antigenic ingredient or ingredients can be mixed with excipients which are pharmaceutically acceptable and compatible with the active ingredient. Examples of suitable excipients are water, saline, dextrose, glycerol, ethanol, or the like, and combinations thereof. In addition, if desired, the vaccine can contain minor amounts of auxiliary substances such as wetting or emulsifying agents, pH buffering agents, or adjuvants such as aluminium hydroxide or muramyl dipeptide or variations thereof. In the case of peptides, coupling to larger molecules (e.g. KLH or tetanus toxoid) sometimes enhances immunogenicity. The vaccines are conventionally administered parenterally, by injection, for example, either subcutaneously or intramuscularly. Additional formulations which are suitable for other modes of administration includes suppositories and, in some cases, oral formulations.

The vaccines of the invention also include nucleic acid vaccines comprising nucleic acid molecules encoding the human retroviral Sag or modified forms of the SAg known to be immunogenic but no longer active as SAgs. The nucleic acid vaccines, particularly DNA vaccines, are usually administered in association with a pharmaceutically acceptable carrier as an intra-muscular injection.

The invention also relates to use of substances inhibiting either the retroviral function or the SAg function of the associated retroviruses, or Sag synthesis, in therapy for HERV-W associated disorders such as MS. These substances may be identified by the screening procedures described herein.

The invention further relates to methods for treatment or prevention of MS comprising administering an effective amount of a substance capable of inhibiting retroviral function or a substance capable of inhibiting SAg activity or synthesis.

Different aspects of the invention are illustrated in the figures:

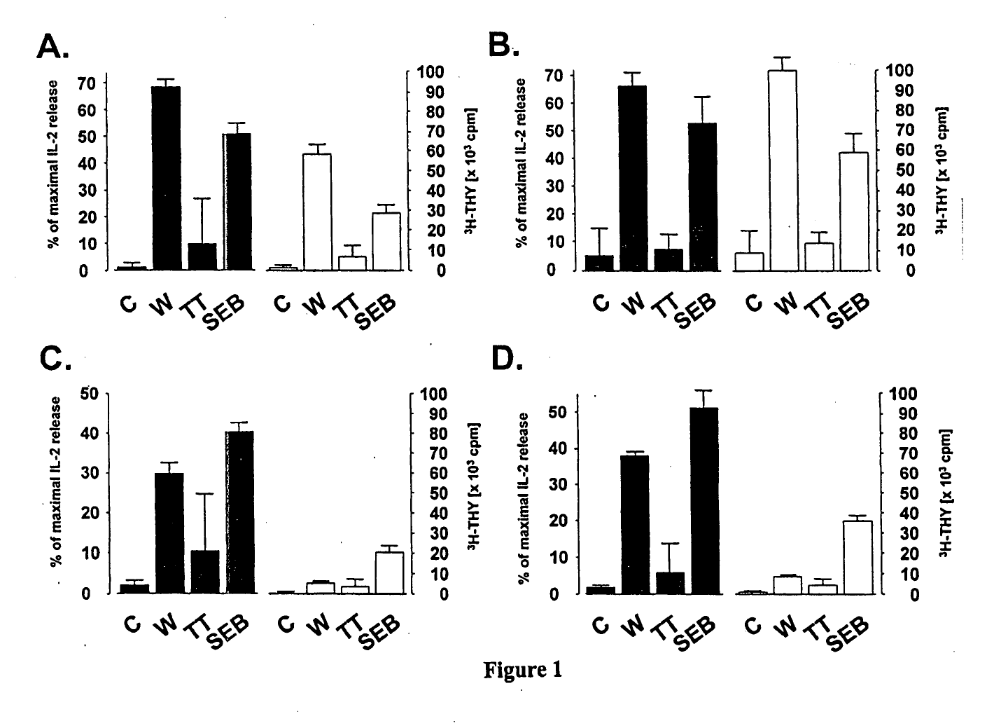

FIG. 1: proliferation assay measured by 3H-thymidine incorporation, and IL2-release assay, measured by IL2 release.

legend: C=pCi (expression vector alone); W=pCi74 (expression vector containing pCl-HERV W-ENV); TT=Tetanus Toxoid; SEB=Staphylococcal enterotoxin B; open bars show ³H-thymidine incorporation; dark bars show IL-2 release.

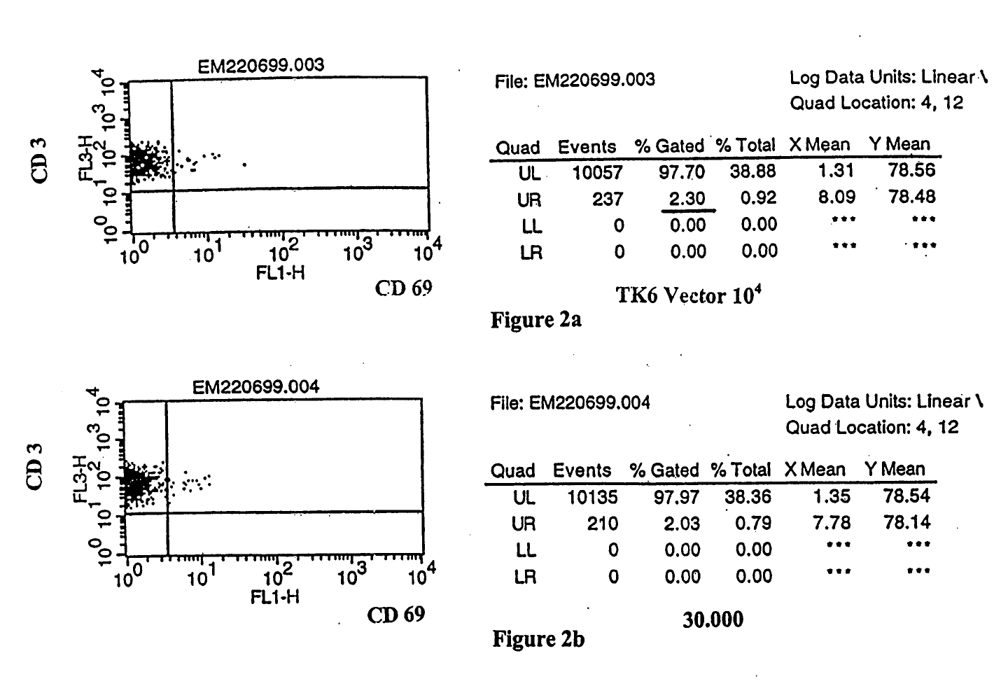

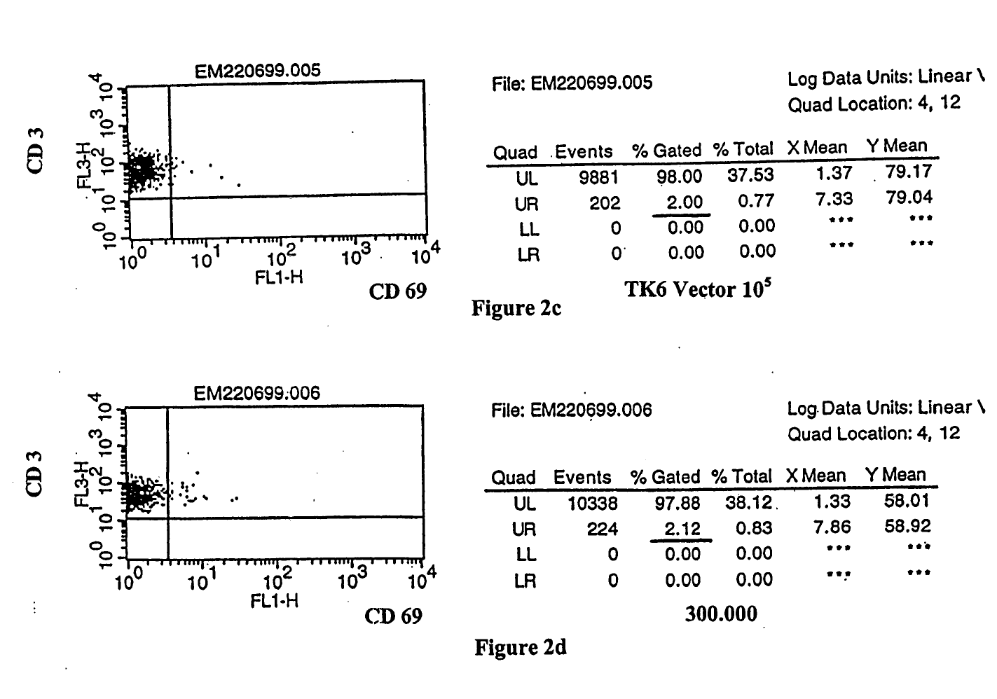

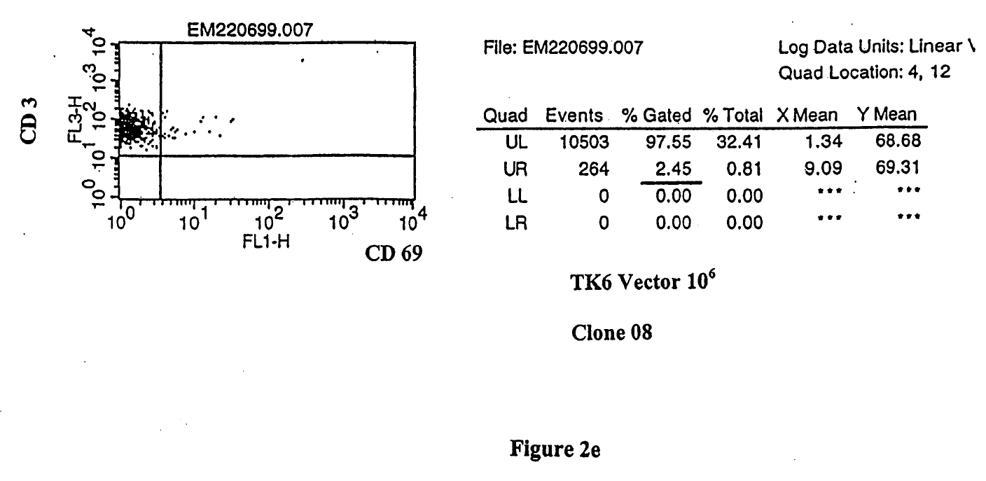

FIG. 2: T-cell activation using CD69 (early T-cell activation marker). Expression vector alone (=“TK6 vector”, also designated TK6 pCl-neo).

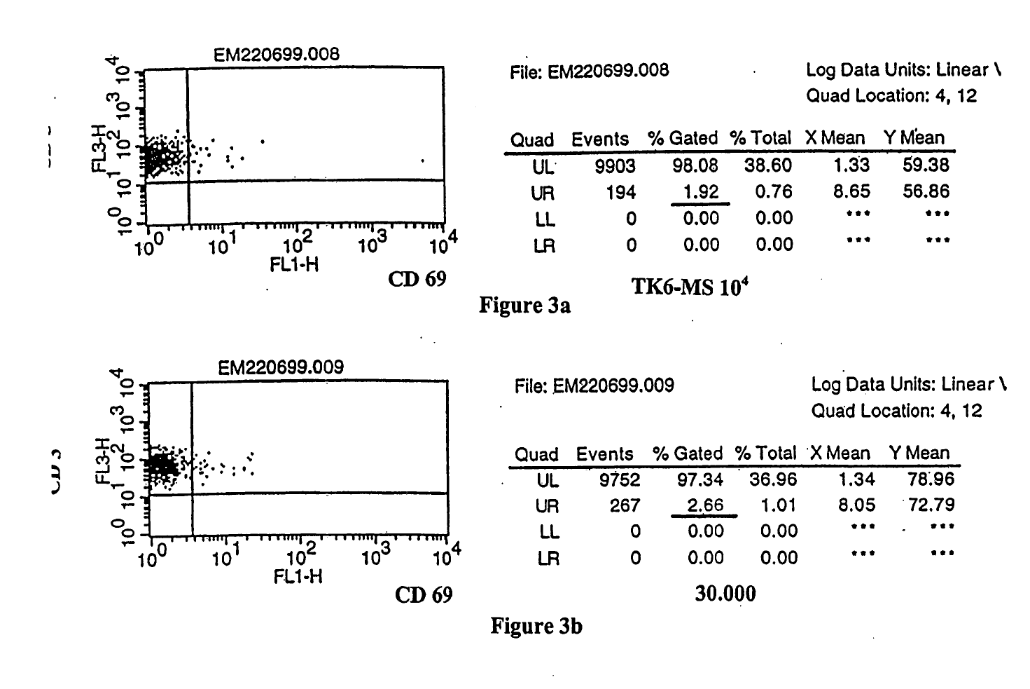

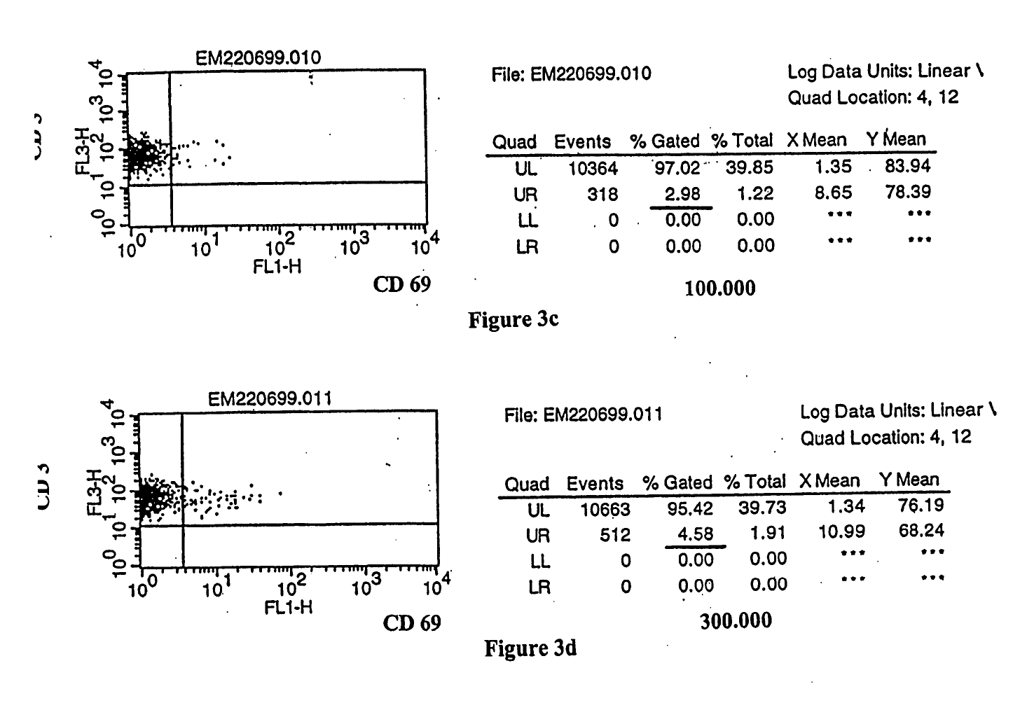

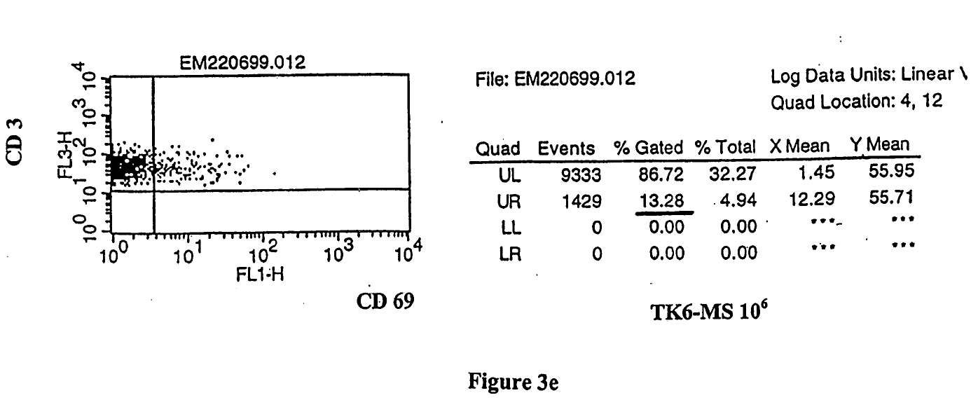

FIG. 3: T-cell activation using CD69 (early T-cell activation marker). Expression vector containing pCl-HERV W-ENV (also designated TK6-MS).

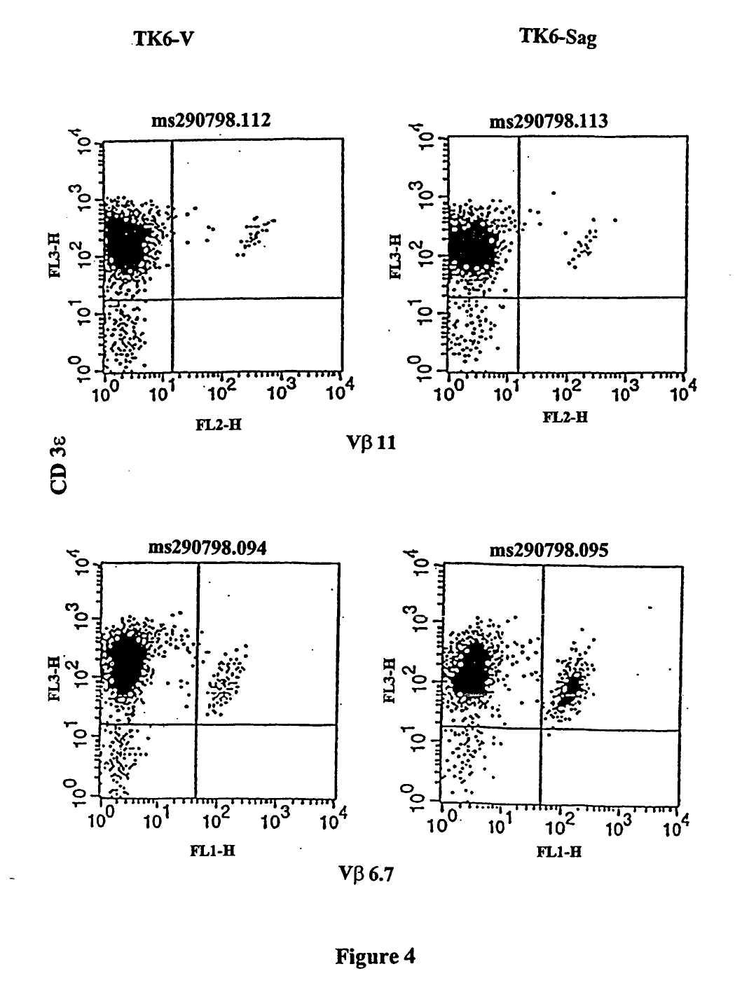

FIG. 4: T-cell enrichment of transfectants showing Vβ6.7 specific enrichment. No specific enrichment of Vβ11⁺ is observed (TK6V=TK6 pClNeo; TK6Sag=TK6-pCl HERV-W ENV)

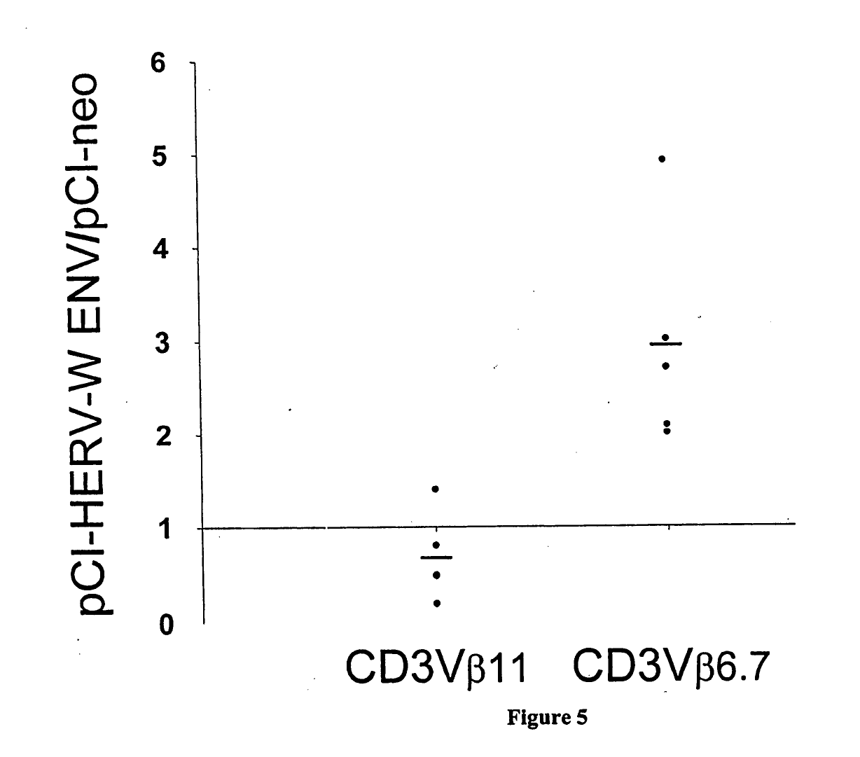

FIG. 5: T-cell enrichment showing Vβ6.7 specific enrichment (results of enrichments shown in FIG. 4); “CD3Vβ11” signifies double positive CD3⁺ and Vβ11⁺; “CD3Vβ6.7” signifies double positive CD3⁺ and Vβ6.7⁺

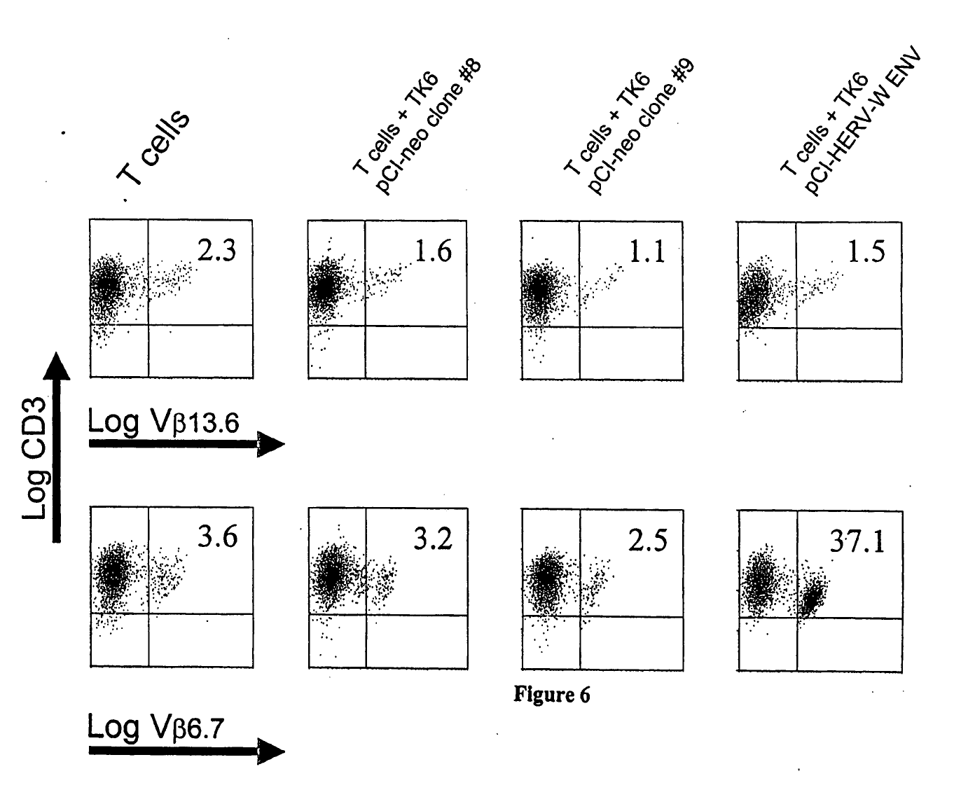

FIG. 6: T-cell enrichment (TK6V=TK6 pClNeo; TK6Sag=TK6-pCl HERV-W ENV; C#8, and C#9 signify clone #8 and #9 respectively)

FIG. 7: Envelope protein of HERV-W (also known as HERV-7q): <<G>> is the full length protein; <<GT>> is the truncated version. Putative Cleavage site SU-TM shown between amino acids 317 and 318.

FIG. 8: Alignment of Envelope protein of HERV-W (also known as HERV-7q ): <<G>> is the full length protein; <<GT>> is the truncated version; with sequences described by Blonde et al (1999) (designated L1 and L2)

FIG. 9: Nucleic acid encoding HERV-W (also known as HERV-7q) env including 5′ UTR and 3′ UTR. Translation initiation codon and stop codon shown in bold type.

FIG. 10: Nucleic acid of coding region of HERV-W (also known as HERV-7q) env.

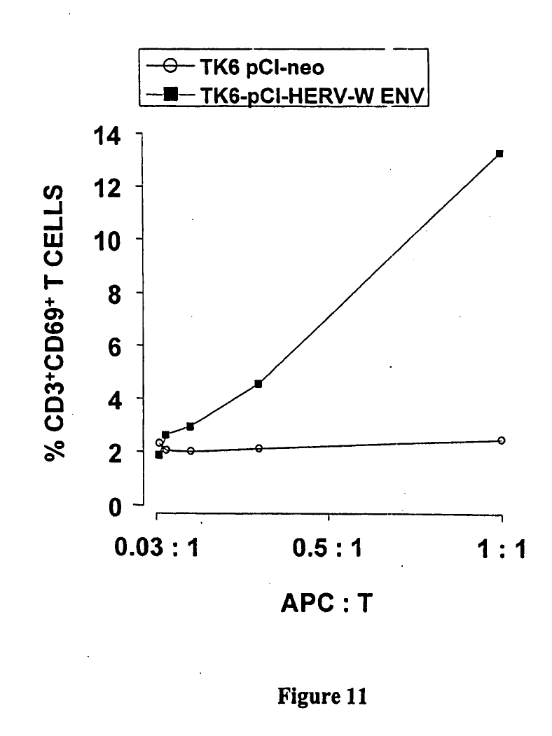

FIG. 11: Summary of results shown in FIGS. 2 and 3, showing mitogenic activity.

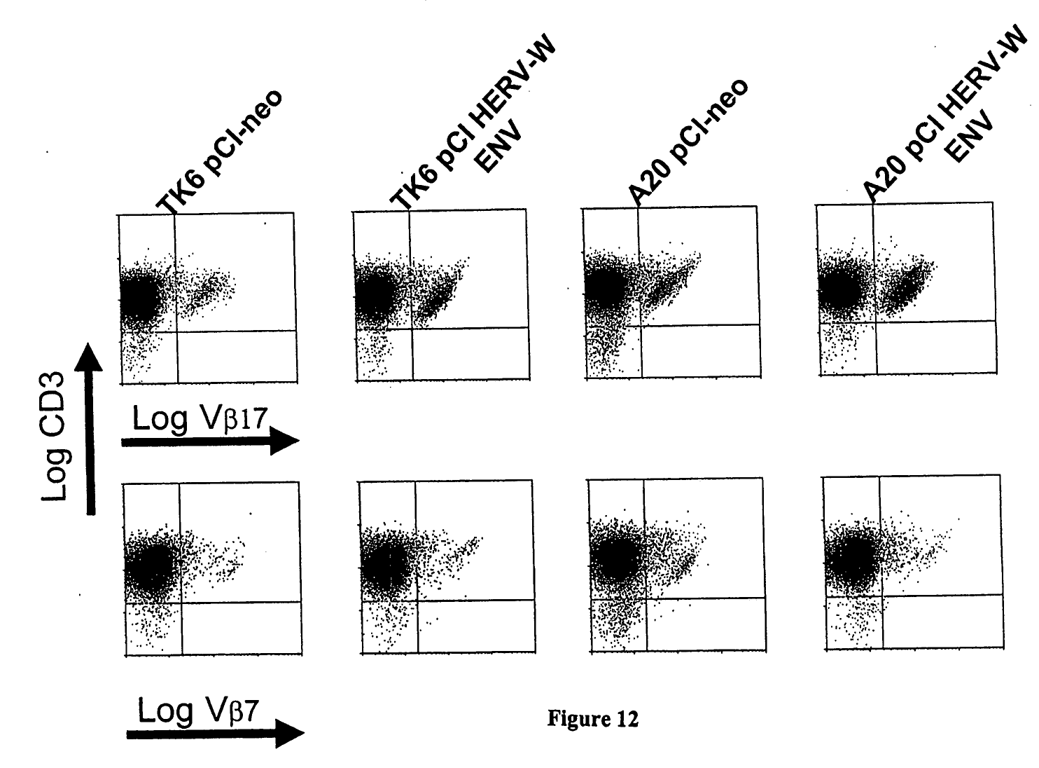

FIG. 12: T-cell enrichment of transfectants in TK6 cells, and mouse A20 cells (null background for HERVs) showing Vβ17 specific enrichment in both cell types. No specific enrichment of Vβ7⁺ is observed (TK6 pClNeo/A20 pCl-neo=TK6 and A20 cells respectively, containing empty expression vector; TK6 pCl HERV-W ENV/A20 pCl HERV-W ENV=TK6 and A20 cells respectively, containing HERV-W ENV)

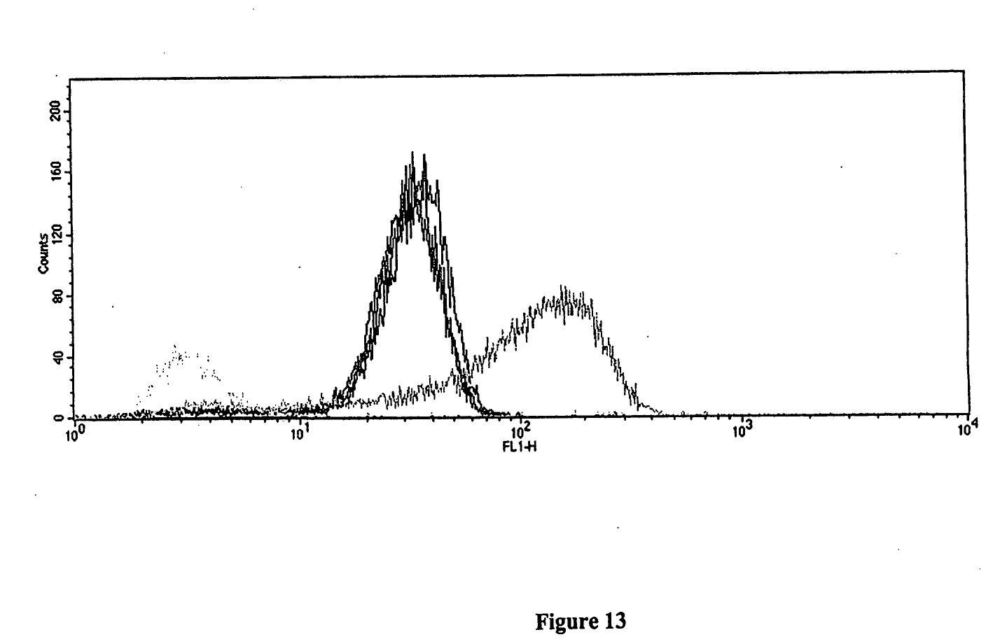

FIG. 13: GFP expression of bicistronic constructs in HtTA4 cells. The left hand curve is a negative control. The remaining curves are expression levels of different HtTA4 transfectant clones containing pCDL-HERV-W ENV. The x-axis is Log EGFP and the y-axis is the number of events.

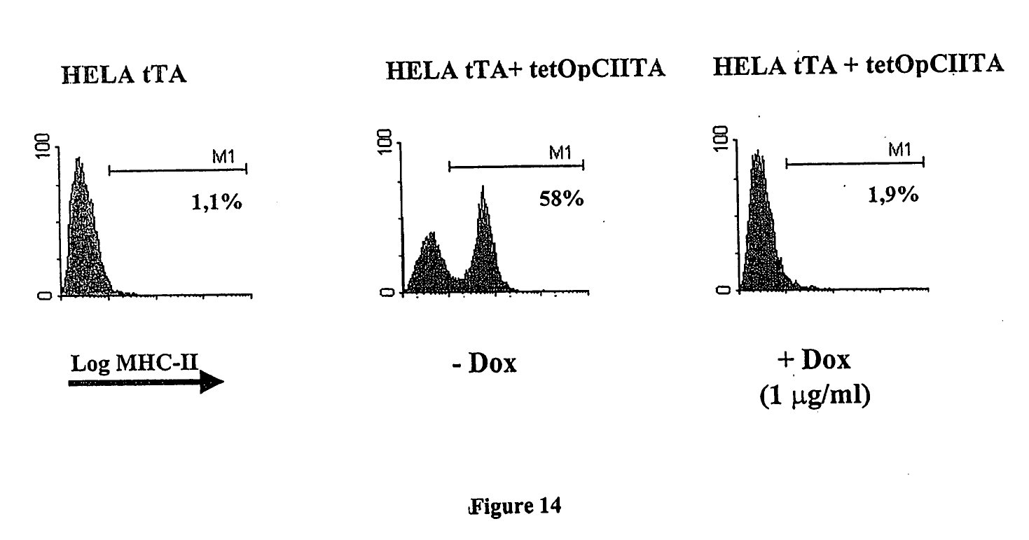

FIG. 14: Repression of MHC-II expression on HELA-tTA cells by dox. HtTA 4 cells express CIITA conditionally in the absence of the repressor Dox. In the presence of Dox (1 μg/ml), no significant expression of CIITA occurs.

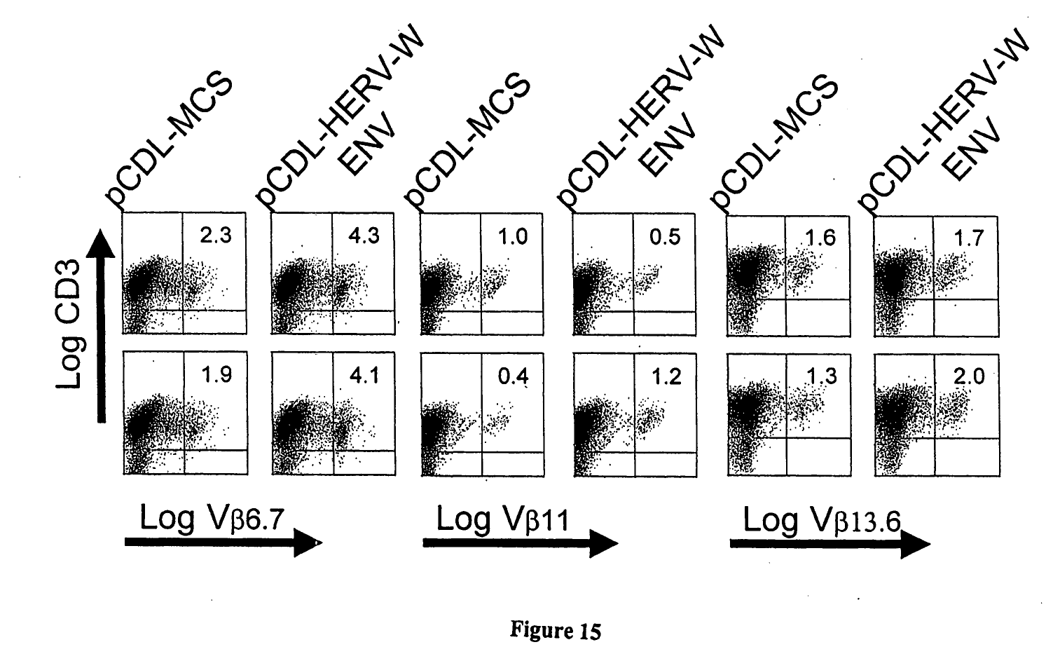

FIG. 15: T-cell enrichment of HtTA 4 cells transfected with bicistronic constructs pCDL-MCS=empty expression vector, pCDl HERV-W ENV=full length env. Specific enrichment of Vβ6.7 is shown. No specific enrichment of Vβ11⁺ or Vβ13.6

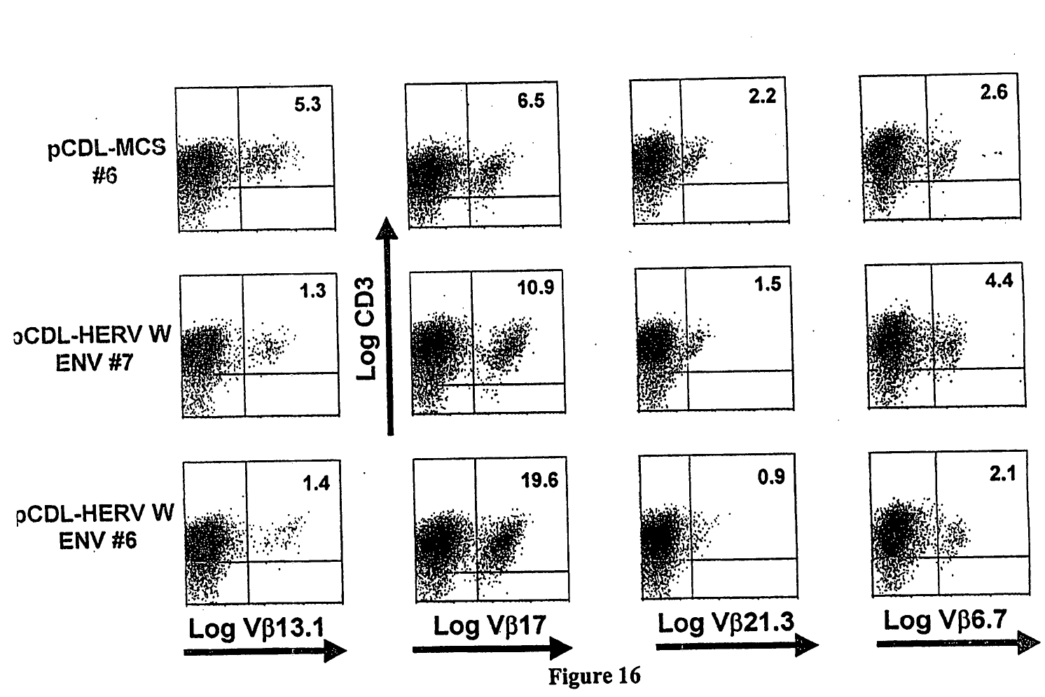

FIG. 16: HtTA transfectants/PMBC donor n°1 at 2 weeks of stimulation, with control pCDL-MCS, clone #6, and HERV-W ENV-expressing bicistronic constructs pCDL-HERV W-ENV, clones #7 and #6.

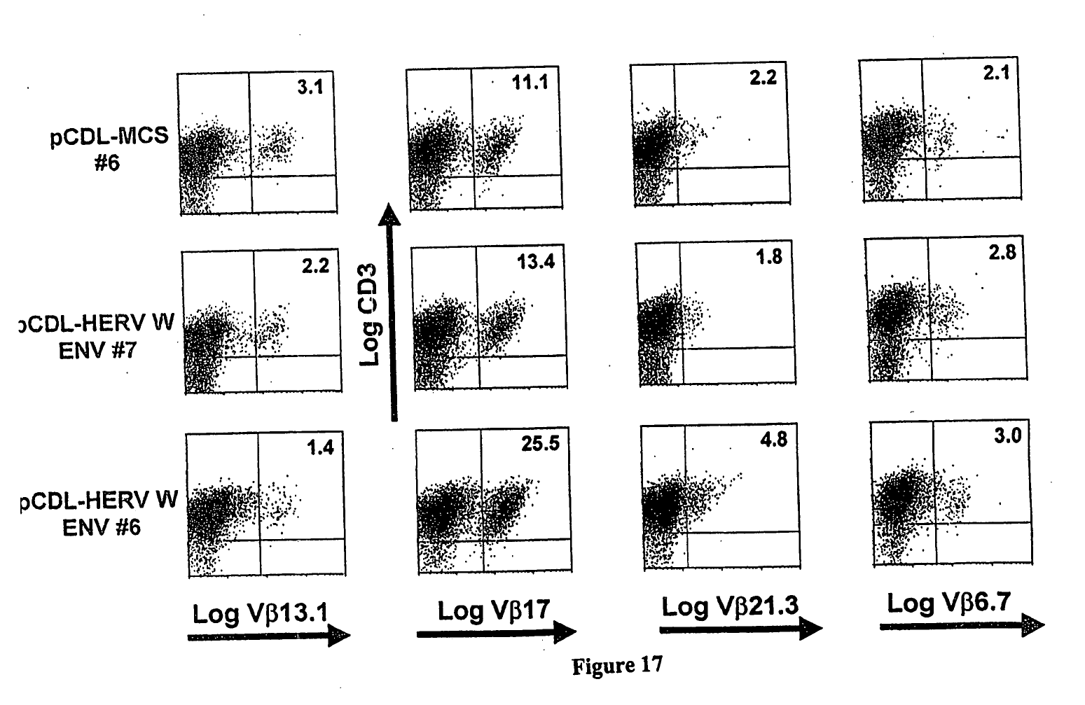

FIG. 17: HtTA transfectants/PMBC donor n°2 at 2 weeks of stimulation, with control pCDL-MCS, clone #6, and HERV-W ENV-expressing bicistronic constructs pCDL-HERV W-ENV, clones #7 and #6.

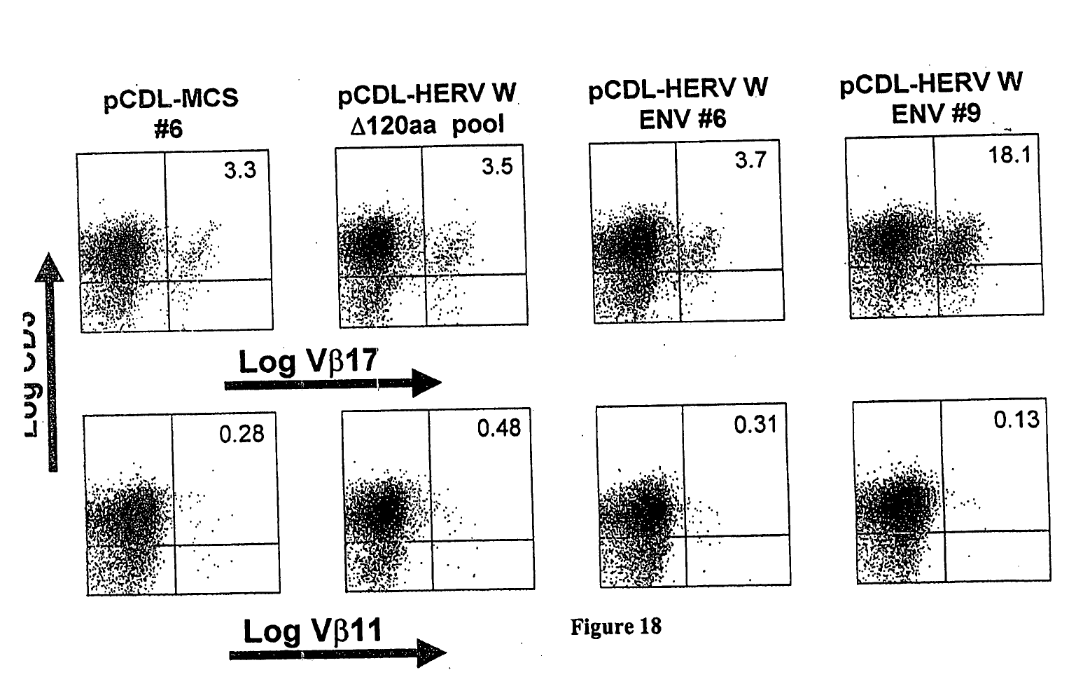

FIG. 18: HtTA transfectants/PMBC donor n°1 at 9 days of stimulation, with control pCDL-MCS, clone #6; HERV-W ENV-expressing bicistronic constructs pCDL-HERV W-ENV, clones #9 and #6, and N-terminal fragment construct pCDL-HERV W-ENV Δ120.

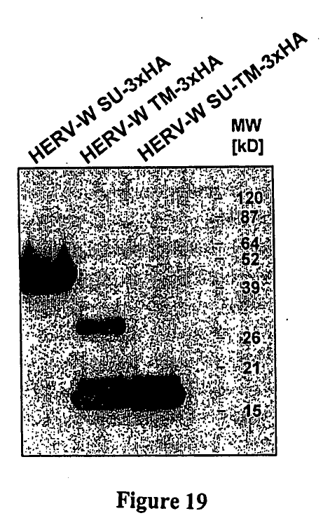

FIG. 19: Western Blot of whole cell extracts of HeLa cells transiently transfected with full length HERV W ENV construct (pCDL-HERV-W SU-TM-3xHA), and SU ans TM sub-units (pCDL-HERV-W SU-3xHA and pCDL-HERV-W TM-3xHA)

FIG. 20: Schematic representaion of bicistronic constructs used in Examples. SRα is a promoter suitable for expression in antigen presenting cells (APCs). “SAg(HA)” represents cistron n°1 comprising the HERV-W-ENV superantigen or sub-unit thereof, linked to a Haemagglutinin tag (HA). The HA tag allows visualization of the expressed ENV protein in Western blots using anti-HA antibodies, and also allows purification of the protein. “IRES” is an internal ribosomal entry site which recrutes ribosomes independently of the presence of a 5′cap. “EGFP” is cistron n°2 comprising enhanced green fluorescent protein. Expression of EGFP allows an indirect measurement of SAg expression in individual clones. “P(A)” is a polyA signal.

### EXAMPLES

1. Molecular Cloning

1.1 HERV Envelope Constructs

The molecular species containing the envelope coding sequence (HERV-W ENV, also designated HERV-7q ENV) has been described (Alliel et al., 1998).

An XbaI-NotI fragment from this species was subcloned into the NheI-NotI linearized expression vector pCI-neo (Promega) to give pCI-74. 3 individual molecular clones from pCI-74 were sequenced and selected for further analysis.

Generation of the minimal coding sequences for the complete envelope, SU-TM, and for the two proteolytical subunits, SU and TM respectively.
i) The predicted minimal envelope coding sequence (SU-TM) was PCR amplified using HERV-W ENV as a template and the primers 5′ATC ggA TCC AAC ACT AgT gCC ACC ATg ggC CTC CCT TAT 3′ and 5′ATT gCg gCC gCT CAg TCg ACT CAT TCA TTC ATC TTT TgT TgC ggg gCT 3′ SEQ ID NOs: 8-9 respectively
   The amplified product was subcloned 5′ blunt-NotI into EcoRV-NotI linearized pBSK (Stratagene) and both strands were sequenced (pBSK74SU-TM).
   The identical procedure was used for the SU and TM coding portions of the envelope coding region of HERV-W.
ii) The primers used to generate pBSK74SU were 5′ATC ggA TCC AAC ACT AgT gCC ACC ATg ggC CTC CCT TAT 3′ and 5′ATT gCg gCC gCT CAg TCg ACT CAT CAT TCA TTC ATC TTT TgT TgC ggg gCT 3′ SEQ ID NOs: 10-11 respectively
iii) The primers used to generate pBSK74TM were: 5′ATC ggA TCC AAC ACT AgT gCC ACC ATg ggC CTC CCT TAT 3′ and 5′ATT gCg gCC gCT CAg TCg ACT CAT TCA TTC AAC TgC TTC CTG CTg CTg AA 3′ SEQ ID NOs: 12-13 respectively

1.2 Construction of Bicistronic Expression Vectors:

Expression cassettes were generated by PCR and sequenced on both strands.

The bicistronic expression vectors were constructed based on pcDL-SRα296: SEQ ID NOs: 14-15 respectively

pcDL: a fragment containing the IRES-EYFP cassette was PCR amplified from pIRES-EYEP (Clontech) with the oligonucleotides 5′ ATT AAT ATC TCG AGA CTA CTG ATC ACG CGT CGA CTC TAG GGC GGC CAA TT 3′ and 5′ CGG GCC TCG AGT TAA TTA ATT ACT TGT ACA GCT CGT CC 3′. Subsequently, the fragment was digested with XhoI and subcloned into pcDL-SRα296, from which the 16S splice junction and the MCS had been previously removed.

Primer sequences used to clone the HERV-W envelope gene into the pCDL vectors (bicistronic expression cassettes): The vector pCi containing the complete HERV-W envelope gene, which was previously employed for SAg assays with monocistronic expression cassettes, was used as template for PCR:

| | |
|---|---|
| SpeEcoTg SU 5′: | |
| ATCACTAGTACGAATTCGCCACCATGGCCCTCCCTTA | SEQ ID NO: 16 |
| TCATATTTTTC | |
| SpeI EcoRI | |
| NotXba SUTM 3′: | |
| GATGCGGCCGCACACGCGTAACTCTAGACTATCTATC | SEQ ID NO: 17 |
| TAACTGCTTCCTGC | |

For the construction of the HA tag SEQ ID NOs: 18-19 respectively pBS-SK-3xHA: 5 μg of each of the following oligonucleotides was resuspended in 100 μl of Tris pH 8.0. 5′ CTA GAG CCA CCA TGG TCG ACG GCT ACC CAT ACG ATG TTC CAG ATT ACG CTG GAT ATC CCT ATG ACG TGC CCG ACT ATG CCG GTT ACC CGT ACG ATG TCC CGG ACT ACG CCG GGC CGC GGT GAT TGA TTG AGC 3′; 5′ GGC CGC TCA ATC AAT CAC CGC GGC CCG GCG TAG TCC GGG ACA TCG TAC GGG TAA CCG GCA TAG TCG GGC ACG TCA TAG GGA TAT CCA GCG TAA TCT GGA ACA TCG TAT GGG TAG CCG TCG ACC ATG GTG GCT 3′. The oligonucleotides were denatured for 5′, annealed and subcloned into pBS-SK-.

2. Cell Lines and Cells

Cell lines were obtained from ATCC: the human B lymphoblast cell line TK6, CRL-8015 and the mouse lymphoma cell line A20 (gentic null background for HERVs), TIB-208. Peripheral blood lymphocytes were generated from blood samples of healthy donors obtained from the blood bank in Geneva by Ficoll Hypaque gradient centrifugation.

HtTA 4: HELA cells stably transfected with the tetoperator-CIITA construct have been previously described (Otten et al., (1998) Eur. J. Immunol. 28, 473-478.)

Transfection: Bulk transfectants of TK6 and A20 cells were generated by electroporation. Cells were split 24 h before transfection and then resuspended at 10×10⁶ cells in 250 μl RPMI with 20 μl (1 μg/μl) linearized plasmid in TE pH 8.0.

Cotransfections: Linearized plasmids encoding either a fusion protein of the hygromycin resistance gene with EGFP or alternative resistance genes, such as blasticidin (BSD, Invitrogen), were cotransfected with the expression vector PBSK74SU-TM, at a molar ratio of 1:10 as compared to the expression vector.

Electroporation was performed at 960 mF, 300 V and infinite resistance, yielding time constants between 60-90 msec. Starting 24 h after transfection, cells were selected for resistance to G418 (50-400 μg/ml) or BSD (1-10 μg/ml) present on the cotransfected plasmid.

Transfection of HtTA4 cells with bicistronic cassettes was carried out with the FUGENE 6 transfection reagent (Roche). Briefly, 100000 cells per well were plated the day before transfection in 6 well plates. 1 μg plasmid DNA was used with 3 μl FUGENE 6 to transfect a 35 mm Patri dish. The percentage of cells transfected was analysed by flow cytometry for GFP expression. For stable transfection of the HtHA4, 1 μg linearized plasmid DNA and 100 ng of linearized blasticidin resistance plasmid were used.

Selection of clones: Bulk transfectants were maintained under continuous drug selection. Individual clones were generated by limiting dilution and selected for by function. Alternatively, bulk cultures of transfectants obtained with bicistronic expression vectors were selected for by FACS sorting for EGFP expression under limiting dilution conditions.

3. Functional Assays
i) Proliferation assays (FIG. 1): transfectants were treated with Mitomycin C (Calbiochem) at 100 μg/ml per 10⁷ cells for 1 hour at 37° C. and washed at least 3 times. 10⁶/ml PBL from healthy blood donors were cultured with transfectants at stimulator: responder ratios of 1:1; 1:3, 1:10 and 1:100 for 48 and 72 hours in 96 round-bottom wells at 37° C., in a final volume of 200 μl. ³H-Thymidine was then added at 1 μCi/well and incorporation measured after 18 hours of incubation at 37° C.
ii) Il-2 release assay (FIG. 1): CTLL-2, ATCC No. TIB-214, was used as indicator cell line. The Il-2 present in supernatants was expressed as % maximal proliferative CTLL-2 response obtained with the highest dose of recombinant human IL-2 (Roche).
iii) T cell activation using CD69 (FIG. 2 and FIG. 3): TK6 cells were transfected with either the expression vector alone (TK6-V, also designated TK6pCl-neo) or with the HERV-W envelope coding sequence (TK6-MS, also designated TK6pCl-HERV-W ENV) and selected for G418 resistance in bulk and maintained under half of the final selecting concentration of G418. Bulk transfectants of TK6-V (=TK6pCl-neo) and TK6-MS (=TK6pCl-HERV-W ENV) were cloned by limiting dilution and selected for SAg (Vβ6.7 enrichment) function first. 10⁶/ml PBL (Ficoll purified peripheral blood lymphocytes from healthy volunteers were obtained from the blood bank in Geneva) were incubated in 24 well plates for 24 hours with 10⁴/ml, 3×10⁴/ml, 10⁵/ml, 3×10⁵/ml and 10⁶/ml of TK6-V (FIG. 2A, B, C, D, E respectively) and TK6-MS (FIG. 3A, B, C, D, E respectively). Cells were then stained with anti CD3 and CD69 antibodies.
   Results are summarized in FIG. 11.
iv) T cell enrichment (FIGS. 4, 5, 6 and 12): After 3 days of specific stimulation the T cells were further expanded in 20 U/ml recombinant IL-2 for 11 days before FACS analysis. Cells were then stained with Vβ antibodies and CD3 (HIT3a/UCHT1), CD4 and CD8 (RPA-T8) antibodies, respectively (all antibodies were from Pharmingen, except where stated). The Vβ antibodies were as follows, the clone designation is in parentheses: Vβ-1 (BL37.2), -2 (MPB2D5), -3 (CH92), -5.1 (IMMU157), -5.2 (36213), -5.3 (3D11), -6.7 (OT145), -7 (ZOE), -8.1 and 8.2 (56C5), -8(a) (16G8), -9 (FIN9), -11 (C21), -12 (VER2.31.1), -12 (S511), -13.1 (IMMU222), -13.6 (JU-74), -14 (CAS1.1.3), -16 (TAMAYA 1.2), -17 (E17.5F3), -18 (BA62), -20 (ELL1.4), -21.3 (IG125), -22 (IMMU 546), -23 (AF 23). A Vβ family was considered to be significantly expanded and enriched if the CD3⁺ (CD4⁺ and CD8⁺, respectively) Vβ⁺ population in a sample was 2 fold increased with respect to the vector control sample. Vβ specificity was assumed to be present when a i) defined Vβ family was at least 2 fold increased with respect to the vector control sample in at least 4 genetically unrelated donors (FIG. 5) and ii) if control Vβ families did not show the equivalent enrichment (Vβ11 in FIG. 4, Vβ11 in FIG. 5, Vβ13 in FIG. 6, Vβ7 in FIG. 12) Results are shown in FIGS. 4, 5, 6 and 12.

It can be concluded from the above that the HERV-W (HERV-7q) superantigen expressed by TK6-11S gives rise to Vβ6.7 and Vβ17 specific enrichment.

v) Functional Assays with Bicistronic Expression Cassettes

A bicistronic expression cassette was generated with IRES driven expression of enhanced green fluorescent protein (EGFP) as indirect marker. First, this serves the purpose of facilitating the structure—function analysis of the SAg. Second, it allows the direct comparison of expression levels of individual constructs.

The following constructs (illustrated schematically in FIG. 20) were generated:
pCDL-MCS empty bicistronic expression cassette.
pCDL-HERV-W ENV bicistronic cassette containing the full length ENV coding sequence (including the signal peptide).
pCDL-HERV-W Δ120 bicistronic cassette containing the sequence coding for the N-terminal 120 amino acid fragment of HERV-W ENV (i.e. only amino acids 1 to 120 of HERV-W ENV, including signal peptide).
pCDL-HERV-W SU-3xHA bicistronic cassette containing the surface protein portion (SU) of HERV-W ENV, including the signal peptide (amino acids 1 to 317 inclusive), and a C-terminal 3xHA tag.
pCDL-HERV-W TM-3xHA bicistronic cassette containing the transmembrane domain (TM) of HERV-W ENV (amino acids 318 to 538 inclusive), and a C-terminal 3xHA tag.
pCDL-HERV-W SU-TM-3xHA bicistronic cassette containing the full surface protein and transmembrane domain (TM) of HERV-W ENV (amino acids 1 to 538 inclusive), and a C-terminal 3xHA tag. This construct corresponds to pCDL-HERV-W ENV with a C-terminal 3xHA tag
   HtTA 4 cells were transfected with the above constructs. Transfectants were selected for comparable EGFP fluorescence (see FIG. 13) and used for T-cell enrichment functional assays as described in section (iv) above.

i) Confirmation of Specific Vβ-6.7, Vβ-17 and, to a Lesser Degree, Vβ-21.3 Expansion Using Bicistronic Constructs:
Results of the T-cell enrichment assays for pCDL-MCS and pCDL-HERV-W ENV, using anti Vβ-6.7, anti Vβ-11 and anti Vβ-13.6 antibodies are shown in FIG. 15. Results are expressed as calculated percentages of double positive CD3⁺/Vβ-6.7 cells, CD3⁺/Vβ-11 cells and CD3⁺/Vβ-13.6 cells. Significant expansion of Vβ-6.7 was observed. No equivalent enrichment was seen with Vβ-11 and Vβ-13.6.
Specific expansion of Vβ-17 and, to a lesser degree, Vβ-21.3 was also demonstrated, as can be seen from the results presented in FIGS. 16 and 17 showing the results obtained with anti Vβ-17, and anti Vβ-21.3 and, for comparison, anti Vβ-13.1.

ii) Vβ Specific T Cell Responses Vary Between Individuals.
In order to define SAg reactivity in different individuals, Vβ-6.7, Vβ-17 and Vβ-21.3 enrichment of peripheral blood lymphocytes (PBL) cultured with stably transfected antigen presenting cells (APCs) was analyzed. A number of healthy blood donors were tested.
Two representative examples are shown, after two weeks of specific stimulation by transfectants, in FIG. 16 (donor 1) and FIG. 17 (donor 2), where Vβ-17⁺ T cells increased 3 fold (donor 1) and 2.3 fold (donor 2). For Vβ-6.7⁺ cells, donor 1 showed an increase of 1.7, whereas donor 2 showed an increase of 1.43. For Vβ-21.3⁺ cells, donor 1 showed a decrease, whereas donor 2 showed an increase of 2.18. The degree of specific expansion (shown as double positive CD3⁺ and Vβ⁺ for the Vβ in question) also varies in a given individual depending upon the clone used i.e. depending upon the expression level of HERV-W ENV.
These results provide evidence that specific Vβ-21.3 and/or Vβ-6.7 and/or Vβ-17 amplification is the result of T cell stimulation by the SAg and that this response varies in genetically distinct individuals. This variability may be accounted for by polymorphic genetic factors and/or immunological tolerance to the SAg. The quantitative character of the stimulation by SAg in an individual is also demonstrated.

iii) Localization of the SAg Activity in the C-Terminal Part of ENV:
HtTA 4 cells were transfected with the bicistronic N-terminal fragment construct pCDL-HERV WΔ120 aa. PBL from a healthy donor were cultured with the thus-obtained stably transfected antigen presenting cells (APCs). After 9 days of specific stimulation, no expansion of Vβ17⁺ T-cells were observed in this individual in response to the pCDL-HERV WΔ120 aa transfectant. In contrast, significant Vβ17⁺ expansion was seen in response to the transfectant expressing the full length construct pCDL-HERV W ENV, clone #9 (see FIG. 18).
These results show that the SAg activity is not contained in the N-terminal 120 amino acids of ENV. At least part of the coding region extending beyond 120 N-terminal amino acids of ENV is therefore indispensable for SAg activity and Vβ expansion.

iv ) Western Blot Analysis:
Whole cell extracts were prepared from HeLa cells (CIITA⁻) transiently transfected with
   pCDL-HERV-W SU-3xHA
   pCDL-HERV-W TM-3xHA
   pCDL-HERV-W SU-TM-3xHA
HA fusion proteins were detected with anti-HA antibodies and revealed with POD-coupled secondary antibodies by chemiluminescence.
The expected sizes of the ENV sub-unit proteins were the following:
   SU-HA: 39 kD
   TM-HA: 28.8 kD
   SU-TM-HA: 63.7 kD
FIG. 19 shows the Western blot. As expected, the SU and TM constructs gave rise to bands at approximately 39 and 28.8 kD. The band at around 20 kD is a small C-terminal fragment carrying the HA tag, of unknown function. No band is seen for the full length envelope SU-TM, showing correct processing, folding and export of ENV outside the cell.

### REFERENCES

CONRAD B. et al, Nature, vol. 371, 351-355, (1994),
CONRAD B. et al, Cell, 90, 303-313, (1997)
PERRON H. et al, Proc. Natl. Acad. Sci. USA, vol 94, 7583-7588, (1997),
ALLIEL Patrick M. et al, C.R. Acad. Sci. Paris, Life Sciences, 321, 857-863, (1998),
BLOND Jean-Luc et al, Journal of Virology, vol. 73 n° 2, p 1175-1185 (1999).

1. An isolated polypeptide having superantigen (SAg) activity, said polypeptide comprising an amino acid sequence consisting of:
i) the amino acid sequence of SEQ ID NO:2; or
ii) the amino acid sequence of SEQ ID NO:4; or
iii) the surface protein portion (SU) of the polypeptide of SEQ ID NO:2, wherein the SU portion of SEQ ID NO:2 consists of residues 21-317, inclusive, of SEQ ID NO:2, or,
iv) the SU portion of the polypeptide of SEQ ID NO:4, wherein the SU portion of SEQ ID NO:2 consists of residues 31-120, inclusive, of SEQ ID NO:4; or
v) the surface protein (SU) and transmembrane portion (TM) of the polypeptide of SEQ ID NO:2, wherein the SU portion and TM portion of SEQ ID NO:2 consist of residues 21-538, inclusive, of SEQ ID NO:2, or
vi) the transmembrane portion (TM) of the polypeptide of SEQ ID NO:2, wherein the TM portion of SEQ ID NO:2 consists of residues 318-538, inclusive, of SEQ ID NO:2, or
vii) a protein having at least 98% identity with protein (i), (ii), (iii), (iv), (v) or (vi), or
viii) a protein fragment consisting of at least 100 consecutive amino acids of protein (i), (ii), (iii), (iv), (v), (vi) or (vii).

2. An isolated polypeptide having superantigen (SAg) activity, said polypeptide having the following formula:
N(a)ₓ-(b)_{y}-(c)_{z}C
wherein
(a) is an amino acid residue, or a sequence of two or more amino acid residues, with the proviso that said sequence does not consist of any one of the signal sequences:
MALPYHIFLFTVLLPSFTLT (SEQ ID NO:5),
MGLPYHIFLCSVLSPCFTLT (SEQ ID NO:6),
MALPYHIFLFTVVSPSFTLT (SEQ ID NO:7)
(b) is a protein or peptide according to claim 1;
(c) is an amino acid residue, or a sequence of two or more amino acid residues
<<x>>=0 or 1,
<<z>>=0 or 1,
provided that (x+z)≧1;
<<y>>≧1;
and N and C indicate amino and carboxy terminals respectively.

3. The isolated polypeptide of claim 2, wherein (b) is a fragment consisting of a stretch of at least 100 consecutive amino acids comprised within amino acids 121 to 538 of SEQ ID NO:2.

4. The isolated polypeptide of claim 3, wherein (b) is:
a fragment consisting of amino acids 121 to 538 of the polypeptide of SEQ ID NO:2, or
a fragment consisting of amino acids 121 to 317 of the polypeptide of SEQ ID NO:2, or
a fragment consisting of amino acids 121 to 350 of the polypeptide of SEQ ID NO:2, or
a fragment consisting of amino acids 121 to 520 of the polypeptide of SEQ ID NO:2.

5. The isolated polypeptide according to any one of claims 1, 2, 3, and 4, wherein the SAg activity is specific for Vβ6.7, Vβ17, or Vβ21.3-TCR chains or combinations thereof.

6. The isolated polypeptide according to claim 5 wherein the SAg activity is specific for Vβ6 and Vβ17-TCR chains.

7. An isolated polypeptide derived from a polypeptide according to any one of claims 1, 2, 3, and 4, said polypeptide being modified so as to be devoid of SAg activity and being capable of generating an immune response against HERV-W retroviral SAg.

8. The polypeptide of claim 7, wherein said polypeptide is denatured, truncated or mutated.

9. The polypeptide of claim 8, wherein said polypeptide is mutated and wherein said mutation comprises deletion, insertion or replacement of at least one amino acid.

10. An isolated polypeptide having superantigen (SAg) activity, comprising a polypeptide having the formula 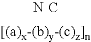
wherein
(a) is an amino acid sequence comprising residues 1-20, inclusive, of SEQ ID NO:2 or a fragment thereof having at least ten amino acids
(b) is an amino acid sequence comprising residues 21-317, inclusive, of SEQ ID NO:2 or a fragment thereof having at least one hundred and fifty amino acids;
(c) is an amino acid sequence comprising residues 318-538, inclusive, of SEQ ID NO:2 or a fragment thereof having at least fifty amino acids;
<<x>>=0 or 1,
<<z>>=0 or 1;
<<y>> is an integer≧1
<<n>>=is an integer≧1
and N and C indicate the amino and carboxy terminal respectively,
with the proviso that the protein [(a)ₓ-(b)_{y}-(c)_{z}]ₙ of Formula (II) does not consist of the full length SP-SU-TM HERV-W-ENV polypeptide of SEQ ID NO:2.

11. An isolated polypeptide having superantigen (SAg) activity, comprising a protein of formula (II): 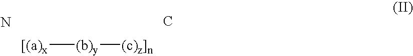
wherein
(a) is an amino acid sequence comprising residues 1-20, inclusive of SEQ ID NO:2 or a fragment thereof having at least five amino acids;
(b) is an amino acid sequence comprising residues 21-317, inclusive, of SEQ ID NO:2 or a fragment thereof having at least one hundred and fifty amino acids;
(c) is an amino acid sequence comprising residues 318-538, inclusive, of SEQ ID NO:2 or a fragment thereof having ten to fifty amino acids;
x is 0 or 1;
z is 0 or 1;
y is an integer>1;
n is an integer>1;
and N and C are the amino and carboxy terminus, respectively,
provided that the protein [(a)ₓ-(b)_{y}-(c)_{z}]ₙ of Formula (II) is not the full length SP-SU-TM HERV-W-ENV polypeptide of SEQ ID NO:2.

12. The polypeptide of claim 11, wherein the polypeptide comprises amino acids 121 to 317 of SEQ ID NO:2.


SEQUENCE LISTING (21 sequences)

SEQ ID NO: 1 - DNA, 2782 bp, Human endogenous retrovirus
atgggagctgttttcatgctatttcactctattaaatcttgcaactgcactcttctggtccatgtttcttacggctcgagctgagcttttgctcaccgtccaccactgctgtttgccaccaccgcagacctgccgctgactcccatccctctggatcctgcagggtgtccgctgtgctcctgatccagcgaagcgcccattgccgctcccaattgggctaaaggcttgccattgttcctgcacggctaagtgcctgggtttgttctaattgagctgaacactagtcactgggttccatggttctcttctgtgacccacggcttctaatagaactataacacttaccacatggcccaagattccattccttggaatccgtgaggccaacgaactccaggtcagagaatacgaagcttgccaccatcttggaagcggcctgctaccatcttggaagtggttcaccaccatcttgggagctctgtgagcaaggaccccccggtgacattttggcgaccaccaacggacatcccaagtgatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatggccctcccttatcatatttttctctgtagtgttctttcaccctgtttcactctcactgcaccccctccatgccgctgtatgaccagtagctcccctcacccagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcaccggggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatgtggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcgaaatcgaagagctttagacttgctaaccgctgagagagggggaacctgtttatttttaggggaagaatgctgttattatgttaatcaatccggaatcgtcactgagaaagttgaagaaattccagatcgaatacaacgtatagcagaggagcttcgaaacactggaccctggggcctcctcagccgatggatgccctggattctccccttcttaggacctctagcagctataatattgctactcctctttggaccctgtatctttgacctccttgttaactttgtctcttccagaatcgaagctgtgaaactacaaatggagcccaagatgcagtccaagactaagatctacggaagacccctggaccggcctgctagcccacgatctgatgttaatgacatcaaaggcacccctcctgaggaaatctcagctgcacaacctctactacgccccaattcagcaggaagcagttagagcggtggtcggccaacctccccaacagcacttaggttttcctgttgagatgggggactgagagacaggactagctggatttcctaggctgactaagaatccttaagcctaggtgggaaggtgaccacatccacctttaaacacggggcttgcaacttagctcacacctgaccaatcagagagctcactaaaatgctaattaggcaaagacaggaggtaaagaaatagccaatcatttattgcctgagagcacagcaggagggacaatgatcgggatataaacccaagttttcgagccggcaacggcaaccccctttgggtcccctccctttgtatgggagctctgttttcatgctatttcactctattaaatcttgcaactgcaaaaaaaaaaaaaaaaaaa

SEQ ID NO: 2 - protein, 538 aa, Human endogenous retrovirus
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 3 - DNA, 1617 bp, Human endogenous retrovirus
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 4 - protein, 120 aa, Human endogenous retrovirus
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGV

SEQ ID NO: 5 - protein, 20 aa, Artificial Sequence
  Description of Artificial Sequence Signal Sequence
MALPYHIFLFTVLLPSFTLT

SEQ ID NO: 6 - protein, 20 aa, Artificial Sequence
  Description of Artificial Sequence Signal Sequence
MGLPYHIFLCSVLSPCFTLT

SEQ ID NO: 7 - protein, 20 aa, Artificial Sequence
  Description of Artificial Sequence Signal Sequence
MALPYHIFLFTVVSPSFTLT

SEQ ID NO: 8 - DNA, 39 bp, Artificial Sequence
  Description of Artificial Sequence HERV-W ENV PCR Primer
atcggatccaacactagtgccaccatgggcctcccttat

SEQ ID NO: 9 - DNA, 48 bp, Artificial Sequence
  Description of Artificial Sequence HERV-W ENV PCR Primer
attgcggccgctcagtcgactcattcattcatcttttgttgcggggct

SEQ ID NO: 10 - DNA, 39 bp, Artificial Sequence; identical to SEQ ID NO: 8
  Description of Artificial Sequence pBSK74SU PCR Primer

SEQ ID NO: 11 - DNA, 51 bp, Artificial Sequence
  Description of Artificial Sequence pBSK74SU PCR Primer
attgcggccgctcagtcgactcatcattcattcatcttttgttgcggggct

SEQ ID NO: 12 - DNA, 39 bp, Artificial Sequence; identical to SEQ ID NO: 8
  Description of Artificial Sequence pBSK74TM PCR Primer

SEQ ID NO: 13 - DNA, 50 bp, Artificial Sequence
  Description of Artificial Sequence pBSK74TM PCR Primer
attgcggccgctcagtcgactcattcattcaactgcttcctgctgctgaa

SEQ ID NO: 14 - DNA, 50 bp, Artificial Sequence
  Description of Artificial Sequence pIRES-EYFP PCR Primer
attaatatctcgagactactgatcacgcgtcgactctagggcggccaatt

SEQ ID NO: 15 - DNA, 38 bp, Artificial Sequence
  Description of Artificial Sequence pIRES-EYFP PCR Primer
cgggcctcgagttaattaattacttgtacagctcgtcc

SEQ ID NO: 16 - DNA, 48 bp, Artificial Sequence
  Description of Artificial Sequence SpeEcoTg SU 5′
atcactagtacgaattcgccaccatggccctcccttatcatatttttc

SEQ ID NO: 17 - DNA, 51 bp, Artificial Sequence
  Description of Artificial Sequence NotXba SUTM 3′
gatgcggccgcacacgcgtaactctagactatctatctaactgcttcctgc

SEQ ID NO: 18 - DNA, 132 bp, Artificial Sequence
  Description of Artificial Sequence pBS-SK-3xHA oligonucleotides for the construction of the HA tag
ctagagccaccatggtcgacggctacccatacgatgttccagattacgctggatatccctatgacgtgcccgactatgccggttacccgtacgatgtcccggactacgccgggccgcggtgattgattgagc

SEQ ID NO: 19 - DNA, 132 bp, Artificial Sequence
  Description of Artificial Sequence pBS-SK-3xHA oligonucleotides for the construction of the HA tag
ggccgctcaatcaatcaccgcggcccggcgtagtccgggacatcgtacgggtaaccggcatagtcgggcacgtcatagggatatccagcgtaatctggaacatcgtatgggtagccgtcgaccatggtggct

SEQ ID NO: 20 - protein, 538 aa, Retrovirus
MGLPYHIFLCSVLSPCFTLTAPPPCRCMTSSSPHPEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTGVHGTSSPYKGLDLSKLHETLRTHTRLVSLFNTTLTGLHEVSAQNPTNCWICLPLNFRPYVSIPVPEQWNNFSTEINTTSVLVGPLVSNVEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLRNRRALDLLTAERGGTCLFLGEECCYYVNQSGIVTEKVEEIPDRIQRIAEELRNTGPWGLLSRWMPWILPFLGPLAAIILLLLFGPCIFDLLVNFVSSRIEAVKLQMEPKMQSKTKIYRRPLDRPASPRSDVNDIKGTPPEEISAAQPLLRPNSAGSS

SEQ ID NO: 21 - protein, 538 aa, Retrovirus
MALPYHIFLFTVVSPSFTLTAPPPCRCMTSSSPYQEFLWRMQRPGNIDAPSYRSLCKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMAIYTEQDLYSYVISKPRNKRVPILPFVIGAGVLGALGTGIGGITTSTQFYYKLSQELNGDMERVADSLVTLQDQLNSLAAVVLRNRRALDSLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS